(19) United States
(12) Patent Application Publication
(10) Pub. No.: US 2026/0069714 A1
(43) Pub. Date: Mar. 12, 2026

(54) LIPIDOID COMPOUNDS AND RELATED COMPOSITIONS AND USES

(71) Applicant: Poseida Therapeutics, Inc., San Diego, CA (US)

(72) Inventors: Lijun Huang, San Diego, CA (US); Ling Li, San Diego, CA (US); Gopi Nath Vemuri, San Diego, CA (US); Khalid A. Hajj, San Diego, CA (US)

(21) Appl. No.: 19/272,804

(22) Filed: Jul. 17, 2025

Related U.S. Application Data
(63) Continuation of application No. PCT/US2024/012234, filed on Jan. 19, 2024.
(60) Provisional application No. 63/480,815, filed on Jan. 20, 2023.

(51) Int. Cl. A61K 48/00; A61K 9/1271; A61K 9/51; C07C 237/08; C12N 15/88

(52) U.S. Cl. CPC A61K 48/0041; A61K 9/1271; A61K 9/5123; C07C 237/08; C12N 15/88; C07C 2601/14

(57) ABSTRACT

Compositions comprising lipidoid compounds, methods of preparing such compositions, and the use of these compositions in gene delivery applications are disclosed.

### RELATED APPLICATIONS

This application is a continuation of International Application No. PCT/US2024/012234, filed Jan. 19, 2024, which claims the benefit of U.S. Provisional Application No. 63/480,815, filed on Jan. 20, 2023, the contents of each of the foregoing applications are incorporated herein by reference in their entireties.

### SEQUENCE LISTING

The Sequence Listing XML associated with this application is provided electronically in XML format and is hereby incorporated by reference into the specification. The name of the XML file containing the Sequence Listing XML is “000218-0136-101-SL.xml”. The XML file is 20,845 bytes in size, was created on Jul. 17, 2025, and is being submitted electronically via USPTO Patent Center.

### FIELD OF THE INVENTION

The present invention relates generally to lipidoid compounds, compositions containing such compounds, methods of preparing these compounds, and the use of these compositions in gene delivery.

### BACKGROUND OF THE INVENTION

There has been a long-felt but unmet need in the art for compositions and methods for delivering nucleic acids to cells and for genetically modifying cells in vivo, ex vivo and in vitro. Widely accepted gene delivery and genetic modification techniques, such as the use of viral vectors, including AAVs, can cause acute toxicity and harmful side-effects in patients. The present disclosure provides improved compositions, methods and kits for the delivery of nucleic acids to various types of cells in vivo, ex vivo and in vitro. More specifically, the present disclosure provides improved lipid nanoparticle compositions and methods of using the same. These lipid nanoparticle compositions and methods allow for the delivery of nucleic acids to cells with high efficiency and low toxicity. Thus, the compositions and methods of the present disclosure have wide applicability to a diverse number of fields, including gene therapy. Further, these lipid nanoparticles and methods allow for the targeted delivery of nucleic acids to the lungs and lung cells, instead of to the liver which is the target of most developed lipid nanoparticle compositions. Thus, the compositions and methods of the present disclosure also have wide applicability to the development of treatments for a broad spectrum of pulmonary diseases.

### SUMMARY OF THE INVENTION

In some aspects, provided are novel compounds. In one aspect, the novel compound is a compound of Formula (I):

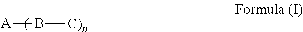

or a salt thereof,
wherein:
A is:

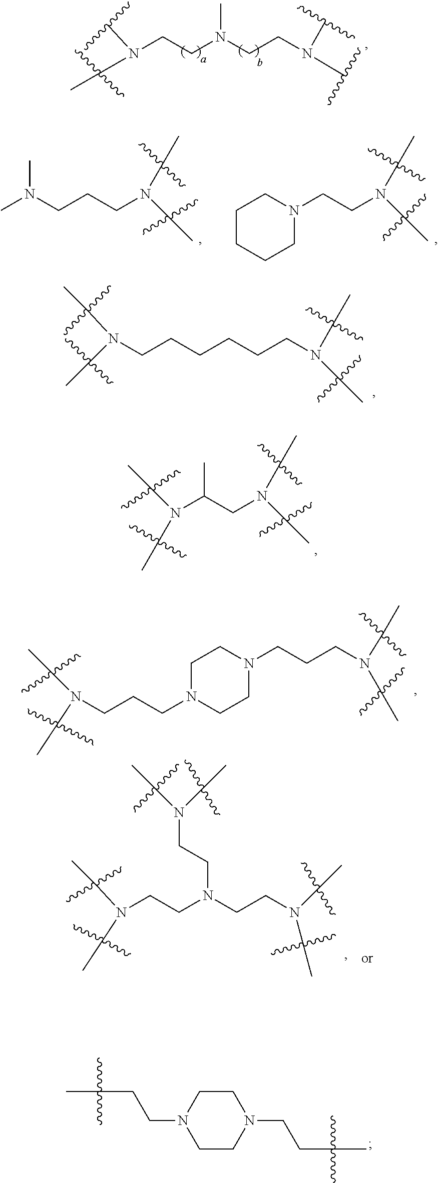

each B is independently:

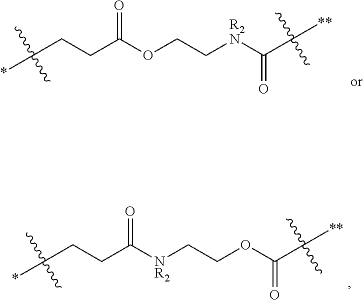

in which * indicates attachment to A and ** indicates attachment to C;
each C is independently:

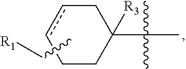

in which [symbol] denotes a single or double bond;
n is an integer between 2 to 6;
a is an integer between 1 to 5;
b is an integer between 1 to 5;
each R₁ is independently C₁-C₁₈ alkyl or C₂-C₁₈ alkenyl;
each R₂ is independently H or methyl; and
each R₃ is independently H or methyl.

In some aspects, provided are novel lipid nanoparticles (“LNPs”) comprising a novel compound. In one aspect, the novel compound is a compound of Formula (I).

In some aspects, provided are pharmaceutical compositions, comprising a composition of the present disclosure and at least one pharmaceutically-acceptable excipient or diluent.

In some aspects, provided are methods of delivering at least one nucleic acid to at least one cell comprising contacting the at least one cell with at least one composition of the present disclosure.

In some aspects, provided are methods of genetically modifying at least one cell comprising contacting the at least one cell with at least one composition of the present disclosure.

In some aspects, provided are methods of treating at least one disease or disorder in a subject in need thereof comprising administering to the subject a therapeutically effective amount of at least one composition of the present disclosure.

In some aspects, provided are methods of delivering at least one nucleic acid to at least one cell comprising contacting the at least one cell with at least one composition of the present disclosure.

In some aspects, provided are cells modified according to methods of the present disclosure.

In some aspects, provided are methods of preferential delivery of at least one composition of the present disclosure to a lung or a lung cell in a subject in need thereof, the method comprising administering to the subject the at least one composition of the present disclosure.

Any of the aspects and/or embodiments described herein can be combined with any other aspect and/or embodiment described herein.

Unless otherwise defined, all technical and scientific terms used herein have the same meaning as commonly understood by one of ordinary skill in the art to which this disclosure belongs. In the Specification, the singular forms also include the plural unless the context clearly dictates otherwise: as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

FIG. 1 shows bioluminescence images of the lungs, liver and spleen of mice following administration of compositions of the present disclosure.

### DETAILED DESCRIPTION OF THE INVENTION

The present disclosure provides novel lipidoid compounds, novel lipid nanoparticle compositions (LNPs) comprising the novel lipidoid compounds, methods for preparing the LNPs, and methods for using same. In a non-limiting example, the compositions and methods of the present limiting disclosure can be used for gene delivery. In a non-limiting example, the compositions and methods of the present disclosure can be broadly used to deliver a nucleic acid to lung cells, in vivo, ex vivo or in vitro, for the treatment of certain diseases and disorders, including, but not limited to lung disorders. In a non-limiting example, the compositions and methods of the present disclosure can be broadly used to deliver a nucleic acid to induce the expression of a secreted therapeutic protein.

### Compositions of the Present Disclosure—Lipid Nanoparticles

The present disclosure provides a composition comprising at least one lipid nanoparticle comprising a compound of the present disclosure and at least one nucleic acid molecule. In some aspects, a lipid nanoparticle can further comprise at least one structural lipid. In some aspects, a lipid nanoparticle can further comprise at least one phospholipid. In some aspects, a lipid nanoparticle can further comprise at least one PEGylated lipid.

### Compounds

In one aspect, the present disclosure provides compounds of Formula (I):

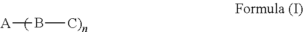

or a salt thereof,
wherein:
A is:

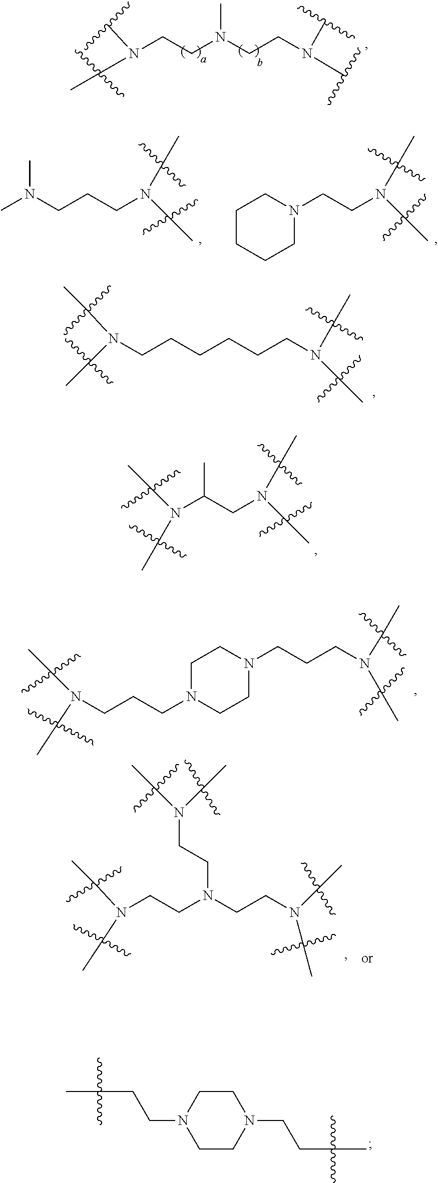

each B is independently:

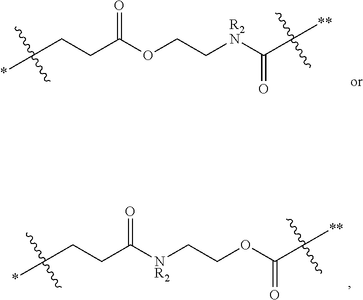

in
which * indicates attachment to A and ** indicates attachment to C;
each C is independently:

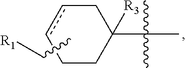

in which [symbol] denotes a single or double bond;
n is an integer between 2 to 6;
a is an integer between 1 to 5;
b is an integer between 1 to 5;
each R₁ is independently C₁-C₁₈ alkyl or C₂-C₁₈ alkenyl;
each R₂ is independently H or methyl; and
each R₃ is independently H or methyl.

In some aspects, A is:

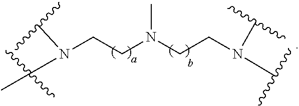

In some aspects, each B is:

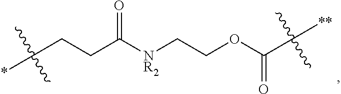

in which * indicates attachment to A and ** indicates attachment to C.

In some aspects, each B is:

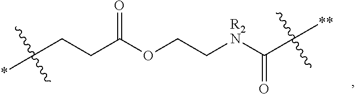

in which * indicates attachment to A and ** indicates attachment to C.

In some aspects, A is

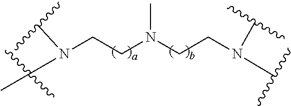

and each B is:

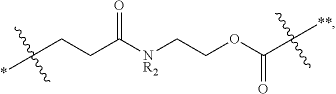

in which * indicates attachment to A and ** indicates attachment to C.

In some aspects, A is

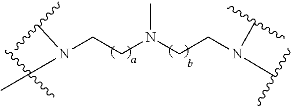

and each B is:

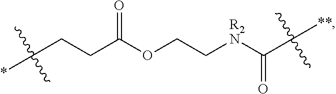

in which * indicates attachment to A and ** indicates attachment to C.

In some aspects, each C is

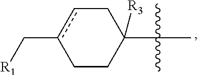

in which [symbol] denotes a single or double bond. In some embodiments, each C is

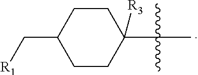

In some embodiments, each C is

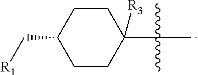

In some embodiments, each C is

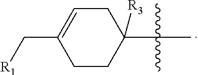

In some aspects, each R₂ is H.

In some aspects, each R₂ is methyl.

In some aspects, each R₃ is H.

In some aspects, each R₃ is methyl.

In some aspects, each R₁ is C₂-C₁₈ alkenyl. In some embodiments, each R₁ is

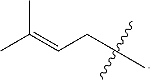 [SMILES: *CC=C(C)C]

In some embodiments, each R₁ is

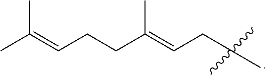 [SMILES: *C/C=C(\C)CCC=C(C)C]

In some aspects, each R₁ is C₁-C₁₈ alkyl. In some embodiments, each R₁ is

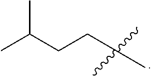 [SMILES: *CCC(C)C]

In some aspects, each R₃ is H and each R₁ is C₂-C₁₈ alkenyl. In some embodiments, each R₁ is

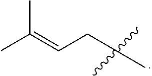 [SMILES: *CC=C(C)C]

In some embodiments, each R₁ is

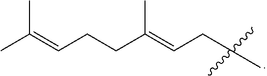 [SMILES: *C/C=C(\C)CCC=C(C)C]

In some aspects, each R₃ is methyl and each R₁ is C₂-C₁₈ alkenyl. In some embodiments, each R₁ is

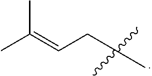 [SMILES: *CC=C(C)C]

In some aspects, each R₃ is H and each R₁ is C₁-C₁₈ alkyl. In some embodiments, each R₁ is

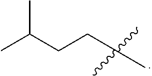 [SMILES: *CCC(C)C]

In some aspects, each R₂ is H, each R₃ is H and each R₁ is C₂-C₁₈ alkenyl. In some embodiments, each R₁ is

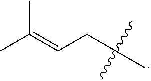 [SMILES: *CC=C(C)C]

In some embodiments, each R₁ is

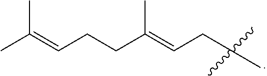 [SMILES: *C/C=C(\C)CCC=C(C)C]

In some aspects, each R₂ is methyl, each R₃ is H and each R₁ is C₂-C₁₈ alkenyl. In some embodiments, each R₁ is

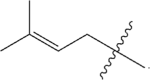 [SMILES: *CC=C(C)C]

In some aspects, each R₂ is H, each R₃ is H and each R₁ is C₁-C₁₈ alkyl. In some embodiments, each R₁ is

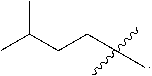 [SMILES: *CCC(C)C]

In some aspects, a is 2.

In some aspects, b is 2.

In some aspects, a is 2 and b is 2.

In some aspects, n is 4.

In some aspects, the compound of Formula (I) is a compound selected from:

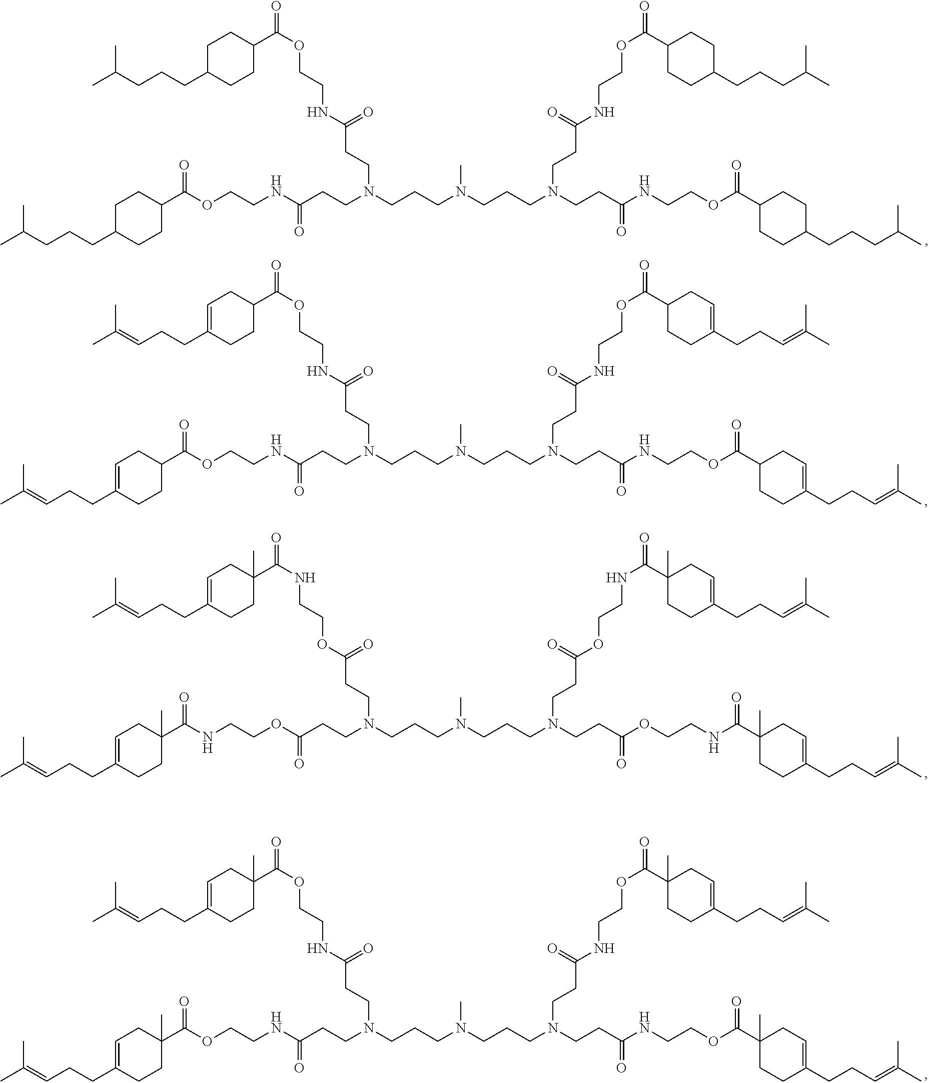

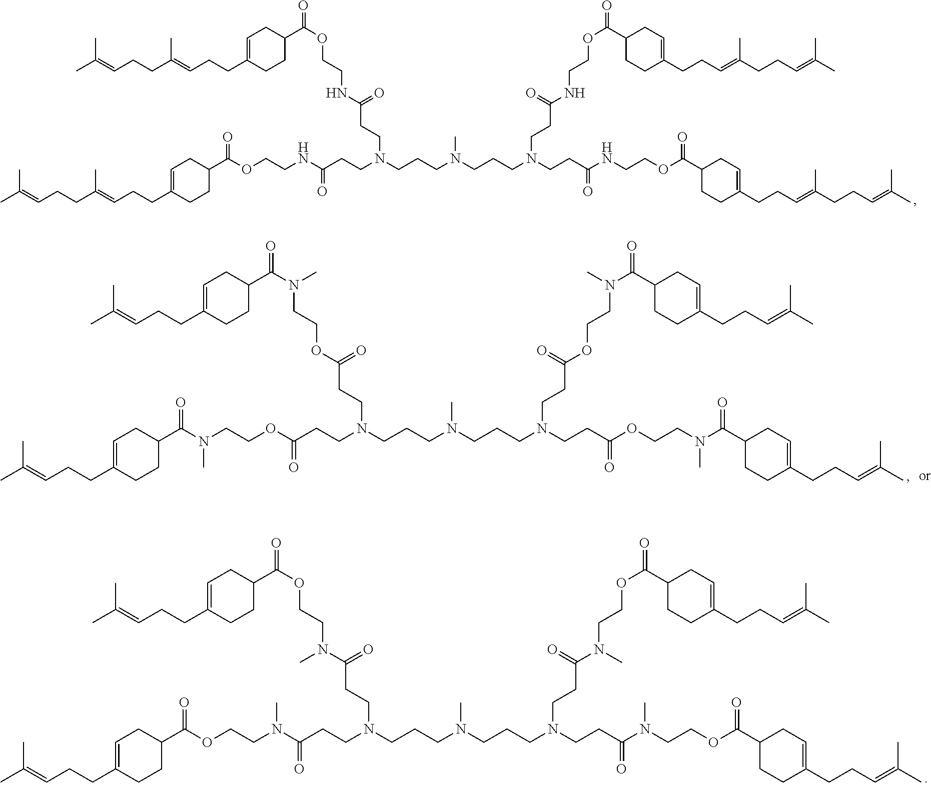

It will be understood that the compounds of any one of the Formulas disclosed herein and any pharmaceutically acceptable salts thereof, comprise stereoisomers, mixtures of stereoisomers, polymorphs of all isomeric forms of said compounds.

It will be understood that while compounds disclosed herein may be presented without specified configuration (e.g., without specified stereochemistry). Such presentation intends to encompass all available isomers, tautomers, regioisomers, and stereoisomers of the compound. In some embodiments, the presentation of a compound herein without specified configuration intends to refer to each of the available isomers, tautomers, regioisomers, and stereoisomers of the compound, or any mixture thereof.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

It will be understood that in any of the formulae described herein, when a “-” is used to indicate linkage between two variables (e.g., A-B), the linkage could be one or more covalent bonds.

General Methods for the Preparation of Compounds of Formula (I) of the Present Disclosure

Compounds of Formula (I) can be prepared using the reagents, intermediates, precursors, methods and schemes disclosed herein or using other commercially available reagents and methods known to those skilled in the art.

### General Procedure for Synthesis of Lipidoids (A)

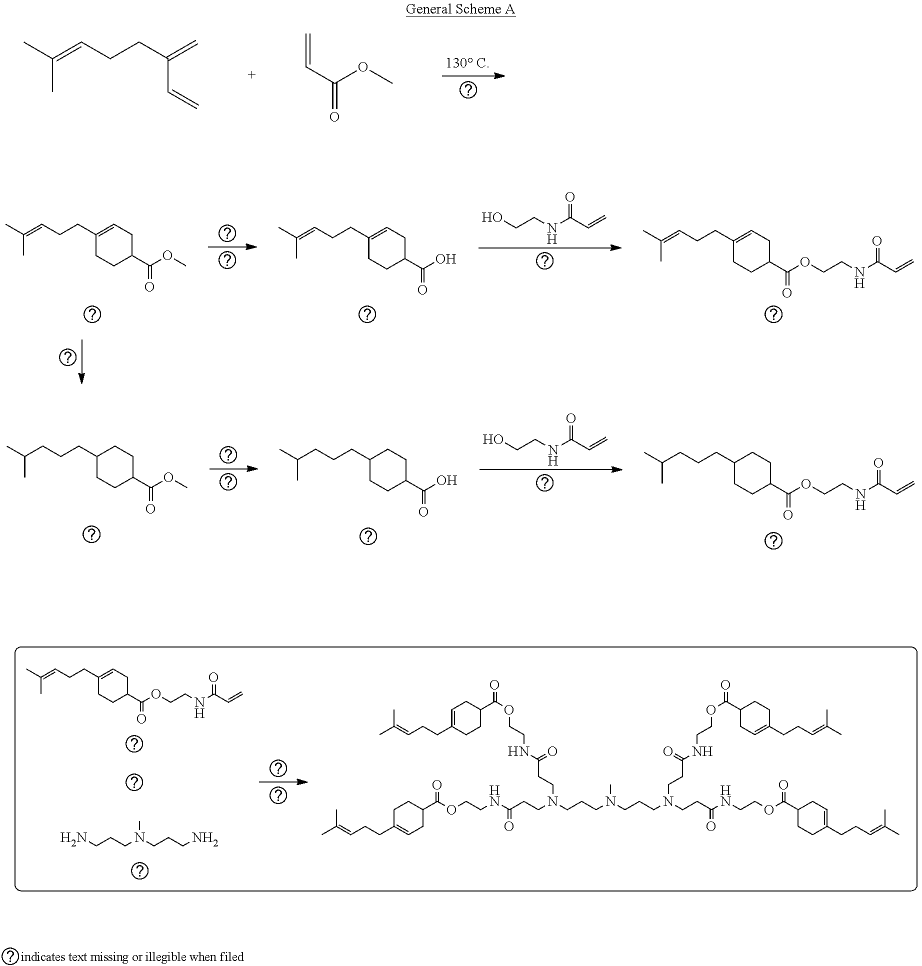

In general, the first step in preparation of compounds of Formula (I) of the present disclosure according to General Scheme A is the reaction of an appropriate terpene, e.g., trans-beta-farnesene, beta-myrcene or other suitable biorenewable terpene, with a suitable unsaturated carboxylic acid derivative, e.g., methyl acrylate, which serves as a dienophile forming a suitable Diels-Alder adduct. For example:

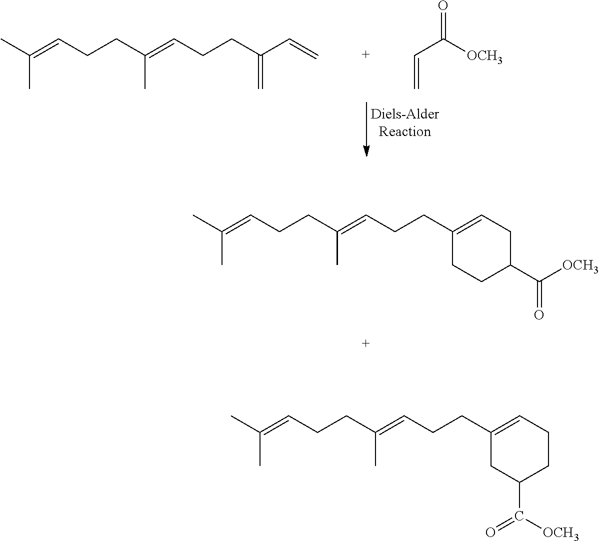

Suitable Diels-Alder products for use in the methods herein include:

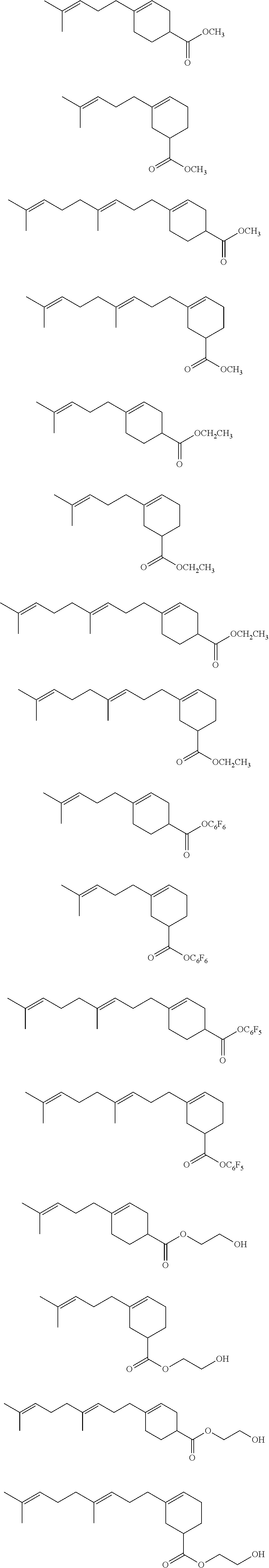

Diels-Alder reactions and Diels-Alder products, such as those used in the preparation of compounds of Formula (I) of the present disclosure according to General Scheme A, are described in WO2022/087175, the contents of which are incorporated by reference in their entirety.

### General Procedure for Diels-Alder Cycloaddition:

Myrcene (7.2 g, 52.8 mmol, 1.0 eq) and methyl acrylate (5.0 g, 58.1 mmol, 1.1 eq) were taken in a 150 mL sealed tube. The tube was then capped and stirred for 16 h at 130° C. The cooled reaction mixture was transferred to a round-bottom flask with CH₂Cl₂ and evaporated. The crude residue was purified by silica gel flash column chromatography with 3% EtOAc/hexane as eluants to afford Myrcene methyl ester (MME). This reaction produces an unequal mixture of 1,3- and 1,4-regioisomers that cannot be separated using conventional column chromatographic methods. This mixture of regioisomers was used in subsequent reactions without isomer separation.

Suitable temperatures are generally between 30° C. and 150° C. Higher temperatures are generally associated with higher yields.

Following the Diels-Alder Reaction, one optional step is hydrogenation which can be carried out as follows:

### Hydrogenation:

In a round-bottomed flask, compound MME was dissolved in mixed solvents of ethanol and CH₂Cl₂ (5:1). To this solution, 10% Pd/C (15% w/w) was added in one portion and the resulting dark suspension was stirred under a hydrogen atmosphere for 20 h at room temperature. The reaction mixture was filtered through celite and rinsed with CH₂Cl₂ (5×). The filtrate was evaporated to give the hydrogenated compound HMME. The resulting residues proceeded to the next step without any purification.

Compounds which were prepared through either Diels-Alder reaction or through hydrogenation can then undergo saponification to provide My-COOH (using products of Diels-Alder reaction) or hydrogenated My-COOH (HMy-COOH) (using products of hydrogenation reaction):

### Saponification:

In a round-bottom flask, MME or HMME (1.0 eq) was added to methanol (5×) and KOH (1.5 eq) in one portion. The resulting suspension becomes clear when stirred at 50° C. for 20 h. The reaction was cooled and water was added. The reaction mixture was extracted with diethyl ether (3×) and the aqueous layer was then acidified with 6N HCl to pH 2-3 and then extracted with ethyl acetate (4×). Combined ethyl acetate extracts were washed with brine; dried over Na₂SO₄; filtered and evaporated.

The saponification reaction can be achieved at lower temperatures with longer wait times, with other bases (e.g., NaOH or LiOH), and in different solvents.

Following saponification, compounds My-COOH or HMy-COOH can then undergo esterification to provide acrylamide compounds 2M or hydrogenated 2M (2HM).

### Esterification:

In a round bottom flask, My-COOH or HMy-COOH (1.0 eq) was dissolved in dichloromethane (10×). EDC-HCl (1.5 eq) and DMAP (0.4 eq) were added and the resulting solution was stirred for 20 mins at ambient temperature. N-hydroxyethyl acrylamide (1.2 eq) was added dropwise to the reaction mixture and the reaction mixture was stirred for 20 h at ambient temperature. Brine (50 mL) was added and extracted with dichloromethane (4×). Combined organic extracts were dried over Na₂SO₄; filtered and evaporated. The crude was purified by silica gel flash column chromatography with 30-40% ethyl acetate/hexane eluants.

The esterification reaction can be achieved with other ester forming catalysts such as DCC, HATU, or HBTU; or in other solvents such as DMF or NMP.

Finally, compounds of Formula (I) of the present disclosure according to General Scheme A can be prepared from these acrylamide compounds through Aza-Michael Addition.

### Aza-Michael Addition:

In a scintillation vial, amine (404) (1.0 eq) and either 2M or 2HM (5.0 eq) were mixed. The vial was capped, and the reaction mixture was stirred for 3 days at 85° C. The cooled reaction crude was purified by silica gel flash column chromatography using 5-10% MeOH/CH₂Cl₂ eluants.

Any suitable amine substrate can be used in General Scheme A above to generate lipidoid compounds of Formula (I) of the present disclosure. For example, the amines shown in the general procedure for the Aza-Michael addition step below can each be combined with acrylamide compound 2M or 2HM to provide lipidoid compounds of the invention:

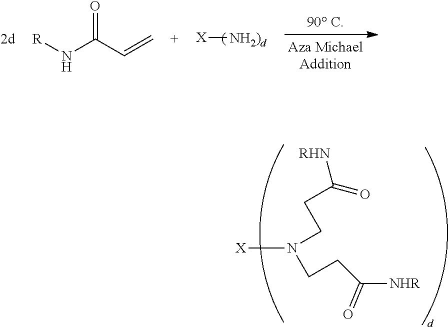

| | |
|---|---|
| 2M | 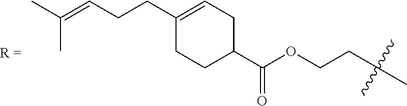 [SMILES: *CCOC(=O)C1CC=C(CCC=C(C)C)CC1] |
| 2HM | 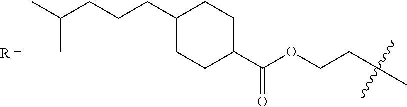 [SMILES: *CCOC(=O)C1CCC(CCCC(C)C)CC1] |
| 201 | 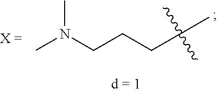 [SMILES: *CCCN(C)C] |
| 202 | 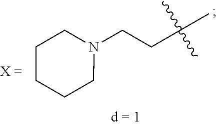 [SMILES: *CCN1CCCCC1] |
| 401 | 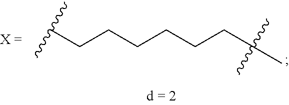 [SMILES: *CCCCCC*] |
| 402 | 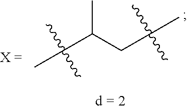 [SMILES: *CC(*)C] |
| 403 | 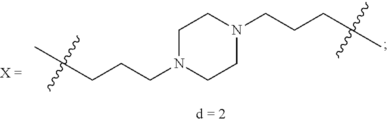 [SMILES: *CCCN1CCN(CCC*)CC1] |
| 404 | 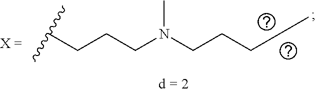 [SMILES: *CCCN(C)CCCCC] |
| 601 | 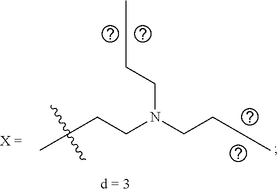 [SMILES: *CCN(CCCC)CCCC] [symbol] |
| Cmpd. 2 | R = 2M; X and d = 404 |
| 2HM-404 | R = 2HM; X and d = 404 |
| 2AM-201 | R = 2M; X and d = 201 |
| 2HM-201 | R = 2HM; X and d = 201 |
| 2AM-202 | R = 2M; X and d = 202 |
| 2HM-202 | R = 2HM; X and d = 202 |
| 2AM-401 | R = 2M; X and d = 401 |
| 2HM-401 | R = 2HM; X and d = 401 |
| 2AM-402 | R = 2M; X and d = 402 |
| 2HM-402 | R = 2HM; X and d = 402 |
| 2AM-403 | R = 2M; X and d = 403 |
| 2HM-403 | R = 2HM; X and d = 403 |
| 2AM-404 | R = 2M; X and d = 404 |
| Cmpd. 1 | R = 2HM; X and d = 404 |
| 2AM-601 | R = 2M; X and d = 601 |
| 2HM-601 | R = 2HM; X and d = 601 |

[symbol]

indicates data missing or illegible when filed

### General Procedure for Synthesis of Lipidoids (B)

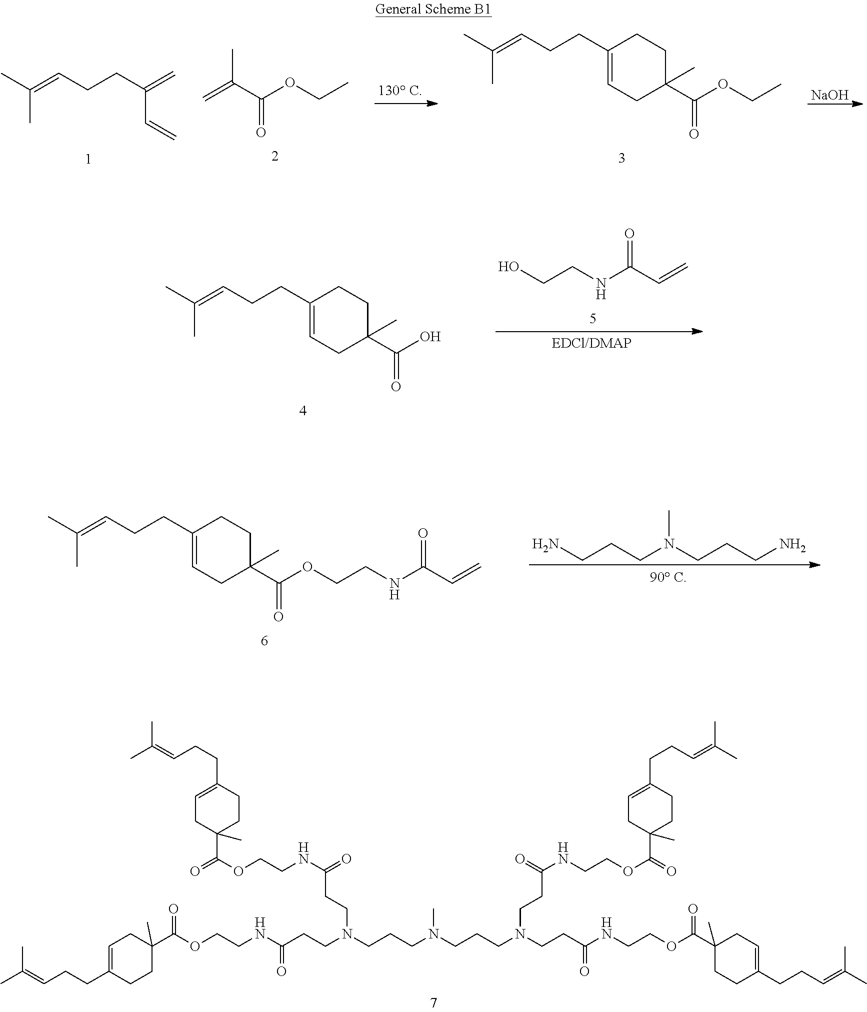

### Intermediate 3

1 (6 g, 53.8 mmol) and 2 (7.34 g, 53.8 mmol) were heated at 130° C. in a sealed tube for 30 hours. The reaction mixture was cooled to room temperature and loaded onto a column (EtOAc/Hexane). Intermediate 3 (6.15 g, 52%) was obtained as a clear oil. ¹H NMR (500 MHz, CDCl₃) δ 5.34 (m, 1H), 5.08 (m, 1H), 4.11 (m, 2H), 2.55-2.39 (m, 1H), 2.13-2.01 (m, 3H), 1.99-1.77 (m, 5H), 1.67 (m, 3H), 1.59 (m, 3H), 1.52-1.46 (m, 1H), 1.23 (t, J=7.1 Hz, 3H), 1.18 (m, 3H).

### Intermediate 4

1N NaOH (90 mL) was added to intermediate 3 (5.1 g, 20.37 mmol) in EtOH (90 mL). The resulting mixture was stirred at 75° C. overnight. 1N HCl was added to make the solution acidic (˜pH2), which was then extracted with ethyl acetate. The combined organic phase was washed with brine, dried over Na₂SO₄ and concentrated. Purification by column chromatography (ethyl acetate/hexane) afforded intermediate 4 (4.37 g, 96%) as a clear oil. ¹H NMR (499 MHz, CDCl₃) δ 5.40-5.31 (m, 1H), 5.07 (m, 1H), 2.52 (m, 1H), 2.14-2.03 (m, 3H), 2.02-1.84 (m, 5H), 1.67 (m, 3H), 1.65-1.50 (m, 4H), 1.21-1.23 (m, 3H).

### Intermediate 6

To 4 (250 mg, 1.12 mmol) in DCM (20 mL) was added 5 (142 mg, 1.23 mmol, 1.1 eq), EDCI (236 mg, 1.23 mmol, 1.1 eq) and DMAP (151 mg, 1.23 mmol, 1.1 eq). The resulting mixture was stirred at room temperature overnight, washed with 1N HCl, saturated NaHCO₃ and brine. The combined organic phase was dried over Na₂SO₄ and concentrated. Purification by column chromatography (ethyl acetate/hexane) afforded intermediate 6 (276 mg, 77%) as a clear oil. ¹H NMR (499 MHz, CDCl₃) δ 6.27 (dd, J=17.0, 1.3 Hz, 1H), 6.12-6.01 (m, 1H), 5.66 (dd, J=10.3, 1.3 Hz, 1H), 5.35 (m, 1H), 5.06 (m, 1H), 4.30-4.15 (m, 2H), 3.60 (q, J=5.5 Hz, 2H), 2.40-2.52 (m, 1H), 2.09-2.02 (m, 3H), 2.00-1.80 (m, 5H), 1.67 (m, 3H), 1.63-1.47 (m, 4H), 1.19-1.20 (m, 3H).

### Product 7

A mixture of N1-(3-aminopropyl)-N1-methylpropane-1,3-diamine (10.5 mg, 0.072 mmol) and 6 (139 mg, 0.43 mmol) was heated at 90° C. for 42 hours. The reaction mixture was cooled to room temperature and loaded onto a column (MeOH/DCM). Product 7 (15 mg, 15%) was obtained as a clear gel. ¹H NMR (499 MHz, CDCl₃) δ 5.33 (m, 4H), 5.06 (m, 4H), 4.14 (t, J=5.8 Hz, 8H), 3.48 (q, J=5.8 Hz, 8H), 2.71 (t, J=6.3 Hz, 8H), 2.52-2.43 (m, 8H), 2.34 (t, J=6.3 Hz, 8H), 2.25 (br, 3H), 2.07-2.01 (M, 8H), 2.00-1.79 (m, 24H), 1.67 (M, 16H), 1.60-1.57 (m, 20H), 1.18-1.17 (m, 12H). MS found 1423.4 [M+H]⁺, calcd for [C83H136N7O12=1423.0]

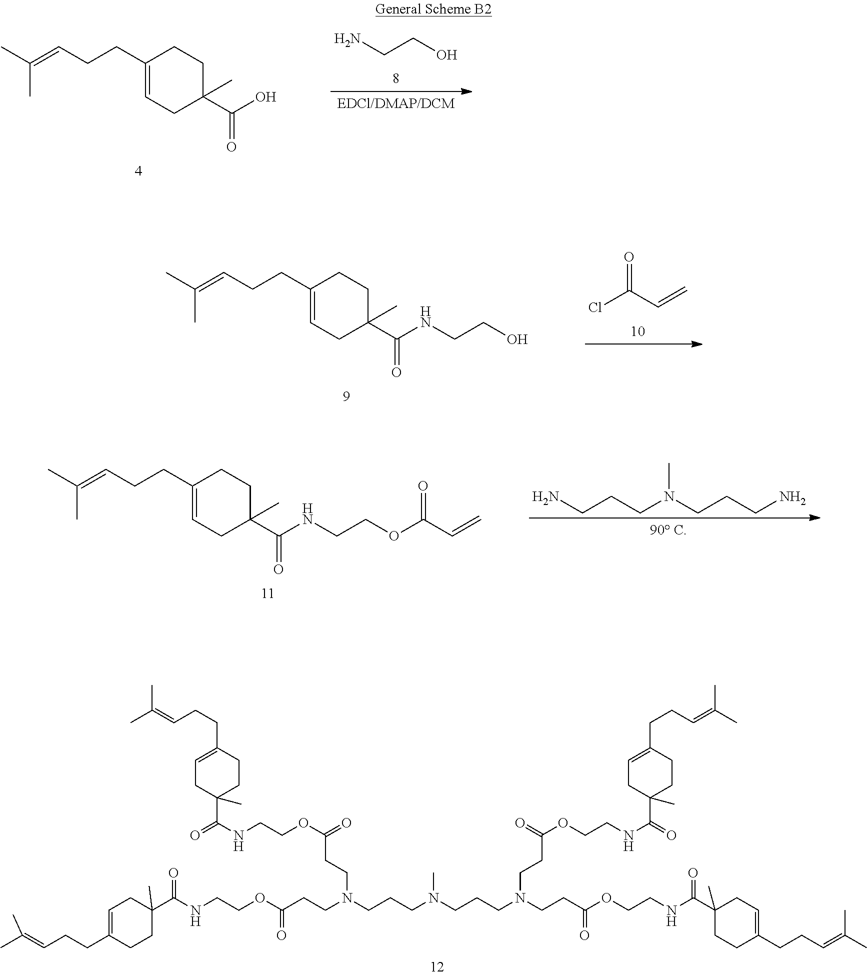

### Intermediate 9

To 4 (1.8 g, 8.1 mmol) in DCM (100 mL) was added 8 (544 mg, 8.9 mmol, 1.1 eq), EDCI (1.7 g, 8.9 mmol, 1.1 eq) and DMAP (1.1 g, 8.9 mmol, 1.1 eq). The resulting mixture was stirred at room temperature overnight, washed with 1N HCl, saturated NaHCO₃ and brine. The combined organic phase was dried over Na₂SO₄ and concentrated. Purification by column chromatography (ethyl acetate/hexane) afforded intermediate 9 (1.77 g, 82%) as a clear oil. ¹H NMR (499 MHz, CDCl₃) δ 6.28-6.21 (m, 1H), 5.46-5.37 (m, 1H), 5.07-5.03 (m, 1H), 3.71-3.67 (m, 2H), 3.49-3.32 (m, 2H), 2.43-2.27 (m, 1H), 2.12-2.04 (m, 3H), 2.01-1.78 (m, 5H), 1.68-1.67 (m, 3H), 1.60-1.59 (m, 3H), 1.58-1.45 (m, 1H), 1.20 (d, J=2.6 Hz, 3H).

### Intermediate 11

To 9 (563 mg, 2.12 mmol) in DCM (30 mL) at 0° C. was added TEA (236 mg, 2.33 mmol, 1.1 eq) and 10 (211 mg, 2.33 mmol, 1.1 eq) dropwise. The resulting mixture was stirred from 0° C. to room temperature overnight, washed with 1N HCl, saturated NaHCO₃ and brine. The combined organic phase was dried over Na₂SO₄ and concentrated. Purification by column chromatography (ethyl acetate/hexane) afforded intermediate 11 (357 mg, 52%) as a clear oil. ¹H NMR (499 MHz, CDCl₃) δ 6.42 (dd, J=17.3, 1.4 Hz, 1H), 6.16-6.09 (m, 2H), 5.86 (dd, J=10.4, 1.3 Hz, 1H), 5.45-5.35 (m, 1H), 5.08-5.03 (m, 1H), 4.25 (m, 2H), 3.61-3.47 (m, 2H), 2.41-2.26 (m, 1H), 2.12-2.02 (m, 3H), 2.01-1.79 (m, 5H), 1.68-1.66 (m, 3H), 1.61-1.45 (m, 4H), 1.17-1.18 (m, 3H).

### Product 12

The mixture of N1-(3-aminopropyl)-N1-methylpropane-1,3-diamine (10.5 mg, 0.072 mmol) and 11 (139 mg, 0.43 mmol) was heated at 90° C. for 42 hours. The reaction mixture was cooled to room temperature and loaded onto a column (MeOH/DCM). Product 12 (23 mg, 23%) was obtained as a clear gel. δ 6.23 (s, 4H), 5.43-5.35 (m, 4H), 5.07 (m, 4H), 4.15 (t, J=5.5 Hz, 8H), 3.53-3.44 (m, 8H), 2.75 (t, J=7.1 Hz, 8H), 2.44 (t, J=7.1 Hz, 12H), 2.39-2.25 (m, 4H), 2.17 (br, 3H), 2.10-2.03 (m, 10H), 2.00-1.93 (m, 16H), 1.91-1.84 (m, 6H), 1.68 (m, 12H), 1.62-1.48 (m, 24H), 1.17 (d, J=4.6 Hz, 12H). MS found 1424.3 [M+H]⁺, calcd for [C83H136N7O12=1423.0]

### General Procedure for Synthesis of Lipidoids (C)

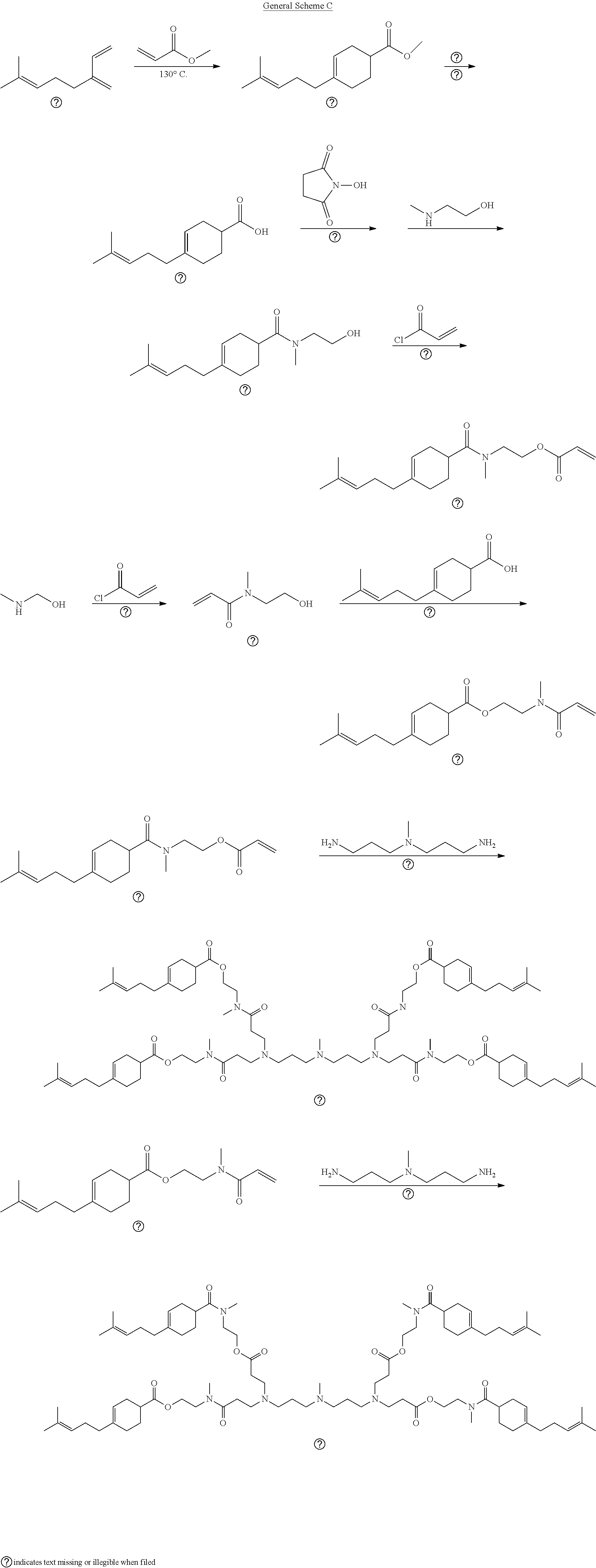

### Intermediate 14

Beta-Myrcene (30 g, 0.22 mol) and methyl acrylate (1.1 eq, 20.86 g) were mixed in a round 250-ml pressure flask and sealed with a screwed cap and the reaction mixture was heated to 130° C. for 48 hrs. After cooling the reaction mixture, the residue was suspended in NaOH in MeOH/H2O (1/1) (15%, 2 eq) and warmed to 50° C. for 3-4 hrs until the starting material was used up as monitored by TLC. The MeOH was removed under reduced vacuum and the remaining residue was extracted with EtOAc and washed with 1 N NaOH three times. The combined aqueous layers were neutralized with 1N HCl to pH 1-2 followed by extraction with EtOAc three times and washing with brine. The crude acid intermediate 14 was obtained as a slightly brown oil, which was used in the next step without purification.

### Intermediate 15

To the solution of intermediate 14 (300 mg, 1.44 mmol) and N—OH succinimide (1.2 eq, 200 mg) was added EDCI (1.5 eq, 413 mg) and DIEA (2.0 eq, 488 μl) at room temperature. The resulting reaction mixture was stirred overnight at room temperature until most of the starting material was used up. The reaction mixture was quenched with sat. NaHCO₃ and extracted with DCM followed by washing and drying. The crude NHS ester was treated with 1-Me amine ethanol (108 mg, 1.0 eq) in dry DMF (3 ml) followed by addition of DIEA (0.36 ml, 1.5 eq). The resulting mixture was stirred at room temperature overnight to get desired amide intermediate 15 (135 mg, 35% yield) after purification by silica gel column (Hex/EtOAc). ¹H NMR (500 MHZ, CDCl₃) of 15: δ5.45-5.44 (m (m, 1H), 5.15-5.08 (m, 1H), 3.80-3.77 (m, 2H), 3.59-3.52 (m, 2H), 3.12 (s, 2.2H), 3.98 (s, 0.8H), 2.72-2.69 (m, 1H), 2.30-2.25 (m, 1H), 2.12-2.03 (m, 5H), 1.97-1.96 (m, 2H), 1.87-1.80 (m, 1H), 1.77-1.73 (m, 1H), 1.66 (s, 3H), 1.62 (s, 3H). MS: m/z 266.2 (M+H).

### Intermediate 16

To the solution of intermediate 15 (105 mg, 0.396 mmol) and DIEA (80 μl, 1.2 eq) in dry DCM (10 ml) was added chloride (51 μl, 1.6 eq) in an ice-bath. The reaction mixture was stirred from 0° C. to room temperature for 20 hrs. The reaction mixture was quenched with 1 N HCl (2 ml) and extracted with DCM, then purified by silica gel column to give intermediate 16 (88 mg) in 70% yield. ¹H NMR (300 MHz, CDCl₃) of 16: δ 6.43-6.39 (m, 1H), 6.15-6.09 (m, 1H), 5.89-5.84 (m, 1H), 5.41-5.42 (m, 1H), 5.15-5.08 (m, 1H), 4.34-4.29 (m, 2H), 3.71-3.64 (m, 2H), 3.11 (s, 1.94H), 3.98 (s, 1.06H), 2.69-2.67 (m, 1H), 2.30-2.27 (m, 1H), 2.12-2.03 (m, 5H), 1.97-1.94 (m, 2H), 1.82-1.75 (m, 1H), 1.71 (s, 3H), 1.60 (s, 3H). MS: m/z 342 (M+Na).

### Product 19

Final product 19 was prepared by Aza-Michael addition in accordance with General Procedure A.

¹H NMR (500 MHZ, CDCl₃) of 19: δ 5.42-5.43 (m, 8H), 5.08-5.11 (m, 8H), 4.23-4.17 (m, 8H), 3.71-3.58 (m, 8H), 3.13-0.91 (m, 12H), 2.71-2.65 (m, 12H), 2.45-2.38 (m, 12H), 2.35-2.25 (m, 8H), 2.27-2.15 (m, 3H), 2.14-2.01 (m, 16H), 2.02-193 (m, 6H), 1.8-1.67 (m, 12H), 1.68 (s, 12H), 1.60 (s, 12H). MS: m/z 1423 (M+1).

### Intermediate 17

To a solution of N-Methyl amino ethanol (2.47 g, 33 mmol) in dry THF (20 ml) was added acryloyl chloride (2.93 ml, 1.1 eq) dropwise at 0° C., and the resulting reaction mixture was stirred from 0° C. to room temperature for 3 hrs. The reaction mixture was quenched with saturated NHCO₃ and extracted with EtOAc to give intermediate 17 (0.65 g, yield 15%) after purification by silica gel column. ¹H NMR (300 MHZ, CDCl₃) of 17: δ 6.71-6.56 (m, 1H), 6.36-6.28 (m, 1H), 5.75-5.64 (m, 1H), 3.8-3.61 (m, 2H), 3.54-3.52 (m, 2H), 3.15 (s, 2H), 3.03 (s, 1H). MS: m/z 130.2 (M+1).

### Intermediate 18

Intermediate 18 was prepared in accordance with the procedures for preparing Intermediate 16.

¹H NMR (500 MHZ, CDCl₃) of 18: δ 6.64-6.55 (m, 1H), 6.37-6.31 (m, 1H), 5.72-5.69 (m, 1H), 5.40-5.37 (m, 1H), 5.10-5.08 (m, 1H), 4.29-4.21 (m, 2H), 3.72-3.64 (m, 2H), 3.14-3.04 (m, 3H), 2.69-2.67 (m, 1H), 2.50-2.48 (m, 1H), 2.22-1.93 (m, 10H), 1.71 (s, 3H), 1.60 (s, 3H). MS: m/z 342 (M+Na).

### Product 20

Final product 20 was prepared by Aza-Michael addition in accordance with General Procedure A.

¹H NMR (500 MHZ, CDCl₃) of 20: δ 5.32-5.37 (m, 8H), 5.09-5.06 (m, 8H), 4.21-4.20 (m, 8H), 3.61-3.58 (m, 8H), 3.07-2.95 (m, 12H), 2.84-2.79 (m, 8H), 2.55-2.45 (m, 16H), 2.30-2.14 (m, 15H), 2.08-1.93 (m, 28H), 1.66 (s, 12H), 1.61 (s, 12H). MS: m/z 1423 (M+1).

### Lipid Nanoparticles of the Present Disclosure

The present disclosure provides lipid nanoparticles (LNPs) comprising one or more compounds of Formula (I). In addition to the one or more compounds of Formula (I), the LNPs of the present disclosure can comprise one or more additional LNP components, as described below:

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one compound of the present disclosure by moles. In some aspects, the at least one compound is at least one compound of Formula (I) as described herein. In some aspects, the at least one compound of the present disclosure is a mixture of two or more compounds of Formula (I).

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least compound of the present disclosure by moles. In some aspects, the at least one compound is at least one compound of Formula (I) as described herein. In some aspects, the at least one compound of the present disclosure is a mixture of two or more compounds of Formula (I).

### Structural Lipids

In some aspects, an LNP can further comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one structural lipid by moles.

In some aspects, an LNP can further comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one structural lipid by moles.

In some aspects, a structural lipid can be a steroid. In some aspects, a structural lipid can be a sterol. In some aspects, a structural lipid can comprise cholesterol. In some aspects, a structural lipid can comprise ergosterol. In some aspects, a structural lipid can be a phytosterol.

In some aspects, the at least one structural lipid is a mixture of two structural lipids.

### Phospholipids

In some aspects, a LNP can further comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one phospholipid by moles.

In some aspects, an LNP can further comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one phospholipid by moles.

As used herein, the term “phospholipid” is used in its broadest sent to refer to any amphiphilic molecule that comprises a polar (hydrophilic) headgroup comprising phosphate and two hydrophobic fatty acid chains. In some aspects, a phospholipid can comprise dioleoylphosphatidylethanolamine (DOPE). In some aspects, a phospholipid can comprise 1,2-Distearoyl-sn-glycero-3-phosphocholine (DSPC). In some aspects, a phospholipid can comprise 1,2-Dioleoyl-sn-glycero-3-phosphocholine (DOPC). In some aspects, a phospholipid can comprise DDPC (1,2-Didecanoyl-sn-glycero-3-phosphocholine), DEPA-NA (1,2-Dierucoyl-sn-glycero-3-phosphate (Sodium Salt)), DEPC (1,2-Dierucoyl-sn-glycero-3-phosphocholine), DEPE (1,2-Dierucoyl-sn-glycero-3-phosphoethanolamine), DEPG-NA (1,2-Dierucoyl-sn-glycero-3[Phospho-rac-(1-glycerol) (Sodium Salt)), DLOPC (1,2-Dilinoleoyl-sn-glycero-3-phosphocholine), DLPA-NA (1,2-Dilauroyl-sn-glycero-3-phosphate (Sodium Salt)), DLPC (1,2-Dilauroyl-sn-glycero-3-phosphocholine), DLPE (1,2-Dilauroyl-sn-glycero-3-phosphoethanolamine), DLPG-NA (1,2-Dilauroyl-sn-glycero-3[Phospho-rac-(1-glycerol) (Sodium Salt)), DLPG-NH4 (1,2-Dilauroyl-sn-glycero-3[Phospho-rac-(1-glycerol) (Ammonium Salt)), DLPS-NA (1,2-Dilauroyl-sn-glycero-3-phosphoserine (Sodium Salt)), DMPA-NA (1,2-Dimyristoyl-sn-glycero-3-phosphate (Sodium Salt)), DMPC (1,2-Dimyristoyl-sn-glycero-3-phosphocholine), DMPE (1,2-Dimyristoyl-sn-glycero-3-phosphoethanolamine), DMPG-NA (1,2-Dimyristoyl-sn-glycero-3[Phospho-rac-(1-glycerol) (Sodium Salt)), DMPG-NH4 (1,2-Dimyristoyl-sn-glycero-3[Phospho-rac-(1-glycerol) (Ammonium Salt)), DMPG-NH4/NA (1,2-Dimyristoyl-sn-glycero-3[Phospho-rac-(1-glycerol) (Sodium/Ammonium Salt)), DMPS-NA (1,2-Dimyristoyl-sn-glycero-3-phosphoserine (Sodium Salt)), DOPA-NA (1,2-Dioleoyl-sn-glycero-3-phosphate (Sodium Salt)), DOPC (1,2-Dioleoyl-sn-glycero-3-phosphocholine), DOPE (1,2-Dioleoyl-sn-glycero-3-phosphoethanolamine), DOPG-NA (1,2-Dioleoyl-sn-glycero-3| Phospho-rac-(1-glycerol) (Sodium Salt)), DOPS-NA (1,2-Dioleoyl-sn-glycero-3-phosphoserine (Sodium Salt)), DPPA-NA (1,2-Dipalmitoyl-sn-glycero-3-phosphate (Sodium Salt)), DPPC (1,2-Dipalmitoyl-sn-glycero-3-phosphocholine), DPPE (1,2-Dipalmitoyl-sn-glycero-3-phosphoethanolamine), DPPG-NA (1,2-Dipalmitoyl-sn-glycero-3[Phospho-rac-(1-glycerol) (Sodium Salt)), DPPG-NH4 (1,2-Dipalmitoyl-sn-glycero-3[Phospho-rac-(1-glycerol) (Ammonium Salt)), DPPS-NA (1,2-Dipalmitoyl-sn-glycero-3-phosphoserine (Sodium Salt)), DSPA-NA (1,2-Distearoyl-sn-glycero-3-phosphate (Sodium Salt)), DSPC (1,2-Distearoyl-sn-glycero-3-phosphocholine), DSPE (1,2-Distearoyl-sn-glycero-3-phosphoethanolamine), DSPG-NA (1,2-Distearoyl-sn-glycero-3[Phospho-rac-(1-glycerol) (Sodium Salt)), DSPG-NH4 (1,2-Distearoyl-sn-glycero-3[Phospho-rac-(1-glycerol) (Ammonium Salt)), DSPS-NA (1,2-Distearoyl-sn-glycero-3-phosphoserine (Sodium Salt)), EPC (Egg-PC), HEPC (Hydrogenated Egg PC), HSPC (Hydrogenated Soy PC), LYSOPC MYRISTIC (1-Myristoyl-sn-glycero-3-phosphocholine), LYSOPC PALMITIC (1-Palmitoyl-sn-glycero-3-phosphocholine), LYSOPC STEARIC (1-Stearoyl-sn-glycero-3-phosphocholine), Milk Sphingomyelin (MPPC; 1-Myristoyl-2-palmitoyl-sn-glycero 3-phosphocholine), MSPC (1-Myristoyl-2-stearoyl-sn-glycero-3-phosphocholine), PMPC (1-Palmitoyl-2-myristoyl-sn-glycero-3-phosphocholine), POPC (1-Palmitoyl-2-oleoyl-sn-glycero-3-phosphocholine), POPE (1-Palmitoyl-2-oleoyl-sn-glycero-3-phosphoethanolamine), POPG-NA (1-Palmitoyl-2-oleoyl-sn-glycero-3[Phospho-rac-(1-glycerol)](Sodium Salt)), PSPC (1-Palmitoyl-2-stearoyl-sn-glycero-3-phosphocholine), SMPC (1-Stearoyl-2-myristoyl-sn-glycero-3-phosphocholine), SOPC (1-Stearoyl-2-oleoyl-sn-glycero-3-phosphocholine). SPPC (1-Stearoyl-2-palmitoyl-sn-glycero-3-phosphocholine), or any combination thereof.

In some aspects, the at least one phospholipid is a mixture of two structural lipids.

### Pegylated Lipid

In some aspects, a LNP can further comprise at least about 0.25%, or at least about 0.5%, or at least about 0.75%, or at least about 1%, or at least about 1.25%, or at least about 1.5%, or at least about 1.75%, or at least about 2%, or at least about 2.25%, or at least about 2.5%, or at least about 2.75%, or at least about 3%, or at least about 3.25%, or at least about 3.5%, or at least about 3.75%, or at least about 4%, or at least about 4.25%, or at least about 4.5%, or at least about 4.75%, or at least about 5%, or at least about 7.5%, or at least about 10% PEGylated lipid by moles.

In some aspects, a LNP can further comprise about 0.25%, or about 0.5%, or about 0.75%, or about 1%, or about 1.25%, or about 1.5%, or about 1.75%, or about 2%, or about 2.25%, or about 2.5%, or about 2.75%, or about 3%, or about 3.25%, or about 3.5%, or about 3.75%, or about 4%, or about 4.25%, or about 4.5%, or about 4.75%, or about 5%, or about 7.5%, or about 10% PEGylated lipid by moles.

As used herein, the term “PEGylated lipid” is used to refer to any lipid that is modified (e.g. covalently linked to) at least one polyethylene glycol molecule. In some aspects, a PEGylated lipid can comprise 1,2-dimyristoyl-rac-glycero-3-methoxypolyethylene glycol-2000, hereafter referred to as DMG-PEG2000.

In some aspects, the at least one PEGylated lipid is a mixture of two PEGylated lipids.

### Exemplary LNP Compositions

The following are exemplary LNP compositions of the present disclosure comprising at least one compound of Formula (I), at least one structural lipid, at least one PEGylated lipid and at least one phospholipid.

In some aspects, a lipid nanoparticle comprising at least one nucleic acid can comprise about 40.75% of at least one compound of Formula (I) by moles, about 51.75% of at least one structural lipid by moles, about 5% of at least one phospholipid by moles, and about 2.5% of at least one PEGylated lipid by moles. In some aspects, a lipid nanoparticle comprising at least one nucleic acid can comprise about 30.75% to about 50.75% of at least one compound of Formula (I) by moles, about 41.75% to about 61.75% of at least one structural lipid by moles, about 0.1% to about 15% of at least one phospholipid by moles, and about 0.1% to about 12.5% of at least one PEGylated lipid by moles. In some aspects, a lipid nanoparticle comprising at least one nucleic acid can comprise about 35.75% to about 45.75% of at least one compound of Formula (I) by moles, about 46.75% to about 56.75% of at least one structural lipid by moles, about 1% to about 10% of at least one phospholipid by moles, and about 1% to about 7.5% of at least one PEGylated lipid by moles.

Table A shows further exemplary LNP compositions of the present disclosure.

TABLE A

| | At least one compound | | | |
|---|---|---|---|---|
| | of Formula (I), | | | |
| | Formula (II) or | | | |
| | Formula (III) | Structural lipid | Phospholipid | PEGylated lipid |
| LNP # | (% by moles) | (% by moles) | (% by moles) | (% by moles) |
| 1 | 40.8 | 51.8 | 5 | 2.5 |
| 2 | about 30.8 to about 50.8 | about 41.8 to about 61.8 | about 0.1 to about 15 | about 0.1 to about 12.5 |
| 3 | about 35.8 to about 45.8 | about 46.8 to about 56.8 | about 1 to about 10 | about 0.2 to about 7.5 |
| 4 | about 38.3 to about 43.3 | about 49.3 to about 54.3 | about 2.5 to about 7.5 | about 1 to about 5 |
| 5 | 40.75 | 51.75 | 5 | 2.5 |
| 6 | about 30.75 to about 50.75 | about 41.75 to about 61.75 | about 0.1 to about 15 | about 0.1 to about 12.5 |
| 7 | about 35.75 to about 45.75 | about 46.75 to about 56.75 | about 1 to about 10 | about 0.2 to about 7.5 |
| 8 | about 38.25 to about 43.25 | about 49.25 to about 54.25 | about 2.5 to about 7.5 | about 1 to about 5 |
| 9 | 40.8 | 51.8 | 5 | 3 |
| 10 | about 30.8 to about 50.8 | about 41.8 to about 61.8 | about 0.1 to about 15 | about 0.1 to about 13 |
| 11 | about 35.8 to about 45.8 | about 46.8 to about 56.8 | about 1 to about 10 | about 0.5 to about 8 |
| 12 | about 38.3 to about 43.3 | about 49.3 to about 54.3 | about 2.5 to about 7.5 | about 1 to about 5.5 |
| 13 | 40.75 | 51.75 | 5 | 3 |
| 14 | about 30.75 to about 50.75 | about 41.75 to about 61.75 | about 0.1 to about 15 | about 0.1 to about 23 |
| 15 | about 35.75 to about 45.75 | about 46.75 to about 56.75 | about 1 to about 10 | about 0.5 to about 8 |
| 16 | about 38.25 to about 43.25 | about 49.25 to about 54.25 | about 2.5 to about 7.5 | about 1 to about 5.5 |

In some aspects, including in the LNP compositions put forth in Table A, the compound of Formula (I) comprised in the LNP composition is one of COMPOUND NOS. 1-7.

In some aspects, including in the LNP compositions put forth in Table A, the structural lipid can be cholesterol.

In some aspects, including in the LNP compositions put forth in Table A, the phospholipid is DOPE.

In some aspects, including in the LNP compositions put forth in Table A, the phospholipid is DSPC.

In some aspects, including in the LNP compositions put forth in Table A, the phospholipid is DOPC.

In some aspects, including in the LNP compositions put forth in Table A, the phospholipid is DPPC.

In some aspects of the preceding LNPs, including the LNP compositions put forth in Tables 1A-1C, the PEGylated lipid is DMG-PEG2000.

In some aspects, including in the LNP compositions put forth in Table A, the structural lipid is cholesterol, the phospholipid is DOPE and the PEGylated lipid is DMG-PEG2000.

In some aspects, including in the LNP compositions put forth in Table A, the structural lipid is cholesterol, the phospholipid is DOPC and the PEGylated lipid is DMG-PEG2000.

In some aspects, including in the LNP compositions put forth in Table A, the structural lipid is cholesterol, the phospholipid is DSPC and the PEGylated lipid is DMG-PEG2000.

In some aspects, including in the LNP compositions put forth in Table A, the structural lipid is cholesterol, the phospholipid is DPPC and the PEGylated lipid is DMG-PEG2000).

### Nucleic Acid Molecules

In some aspects, a lipid nanoparticle of the present disclosure, including those put forth in Tables 1A-1C, can further comprise at least one nucleic acid. In some aspects, a lipid nanoparticle can comprise a plurality of nucleic acid molecules. In some aspects, the at least one nucleic acid or the plurality of nucleic acid molecules can be formulated in a lipid nanoparticle.

Accordingly, a lipid nanoparticle can comprise at least one nucleic acid, at least one compound of the present disclosure, at least one structural lipid, at least one phospholipid, and at least one PEGylated lipid. In some aspects, the lipid nanoparticle can further comprise at least one targeting ligand.

In some aspects, the at least one nucleic acid is a DNA molecule. In one aspect, the at least one DNA molecule is a Doggy Bone DNA molecule. In some aspects, the at least one DNA molecule is a DNA nanoplasmid.

the at least one nucleic acid is an RNA molecule. In some aspects, the RNA molecule is an mRNA molecule. In some aspects, the mRNA molecule further comprises a 5′-CAP. In some aspects, all of the cytidine residues in an mRNA molecule can be 5-methylcytidine.

In some aspects, the at least one RNA molecules is a guide RNA (gRNA) molecule.

In some aspects, an at least one nucleic acid can comprise both mRNA molecules and guide RNA (gRNA) molecules. That is, the LNPs of the present disclosure can comprise both mRNA molecules and gRNA molecules. In some aspects wherein the LNPs comprise both mRNA molecules and gRNA molecules, the mRNA molecules comprise at least one nucleic acid sequence that encodes a fusion protein, wherein the fusion protein comprises: (i) an inactivated Cas9 (dCas9) protein or an inactivated nuclease domain thereof; and (ii) a Clo051 protein or a nuclease domain thereof, and wherein the gRNA molecules encode guide RNA sequence targeting one or more specific genomic loci. In some aspects, the fusion protein can be a Cas-CLOVER protein. In some aspects, the gRNA molecules can target the psk9 gene.

In some aspects wherein the LNPs comprise both mRNA molecules and gRNA molecules, the ratio of mRNA:gRNA can be about 1:2, or about 1:3, or about 1:4, or about 1:5, or about 1:6, or about 1:7, or about 1:8, or about 1:9, or about 1:10 or about 1:1, or about 2:1, or about 3:1, or about 4:1, or about 5:1, or about 6:1, or about 7:1, or about 8:1, or about 9:1 or about 10:1.

In some aspects, an at least one nucleic acid can comprise at least one RNA molecule and at least one DNA molecule. That is, the LNPs of the present disclosure can comprise both RNA molecules and DNA molecules.

In some aspects, the LNPs of the present disclosure can comprise both RNA molecules and DNA molecules, wherein the RNA molecules comprise at least one nucleic acid sequence that encodes a transposase and wherein the DNA molecules comprise at least one nucleic acid sequence that comprises a transposon. In some aspects, the transposase can be any of the transposases described herein. In some aspects, the transposon can be a transposon comprising at least one nucleic acid sequence encoding a therapeutic protein that is to be expressed in the lungs of a subject.

In some aspects wherein the LNPs of the present disclosure comprise both RNA (e.g. mRNA) and DNA, the ratio of RNA to DNA (RNA:DNA) in the LNPs can be about 1:2, or about 1:3, or about 1:4, or about 1:1, or about 2:1, or about 3:1, or about 4:1, or about 5:1, or about 6:1, or about 7:1, or about 8:1, or about 9:1 or about 10:1.

In some aspects, a lipid nanoparticle can comprise lipid and nucleic acid at a specified ratio (weight/weight).

In some aspects, a lipid nanoparticle comprising at least one nucleic acid can comprise lipid and nucleic acid at a ratio of about 5:1 to about 15:1, or about 10:1 to about 20:1, or about 15:1 to about 25:1, or about 20:1 to about 30:1, or about 25:1 to about 35:1 or about 30:1 to about 40:1, or about 35:1 to about 45:1, or about 40:1 to about 50:1, or about 45:1 to about 55:1, or about 50:1 to about 60:1, or about 55:1 to about 65:1, or about 60:1 to about 70:1, or about 65:1 to about 75:1, or about 70:1 to about 80:1, or about 75:1 to about 85:1, or about 80:1 to about 90:1, or about 85:1 to about 95:1, or about 90:1 to about 100:1, or about 95:1 to about 105:1, or about 100:1 to about 110:1, or about 105:1 to about 115:1, or about 110:1 to about 120:1, or about 115:1 to about 125:1, or about 120:1 to about 130:1, or about 125:1 to about 135:1, or about 130:1 to about 140:1, or about 135:1 to about 145:1, or about 140:1 to about 150:1, lipid:nucleic acid, weight/weight.

In some aspects, a lipid nanoparticle comprising at least one nucleic acid can comprise lipid and nucleic acid at a ratio of about 5:1, or about 10:1, or about 15:1, or about 20:1, or about 25:1, or about 30:1, or about 35:1, or about 40:1, or about 45:1, or about 50:1, or about 55:1, or about 60:1, or about 65:1, or about 70:1, or about 75:1, or about 80:1, or about 85:1, or about 90:1, or about 95:1, or about 100:1, or about 105:1, or about 110:1, or about 115:1, or about 120:1, or about 125:1, or about 130:1, or about 135:1, or about 140:1, or about 145:1, or about 150:1, lipid:nucleic acid, weight/weight.

In some aspects, a lipid nanoparticle comprising at least one nucleic acid can comprise lipid and nucleic acid at a ratio of about 10:1, or about 25:1, or about 40:1, lipid:nucleic acid, weight/weight.

In some aspects, a lipid nanoparticle comprising at least one nucleic acid can comprise lipid and nucleic acid at a ratio of about 20:1, or about 40:1, or about 60:1, or about 80:1, or about 120:1 lipid:nucleic acid, weight/weight.

In some aspects, including in the LNPs put forth in Table A, the ratio of lipid to nucleic acid in the nanoparticle can be about 30:1 to about 50:1 (w/w), or about 35:1 to about 45:1 (w/w). In some aspects, the ratio of lipid to nucleic acid in the nanoparticle can be about 40:1 (w/w).

In some aspects, including in the LNPs put forth in Table A, the ratio of lipid to nucleic acid in the nanoparticle can be about 40:1 to about 60:1 (w/w), or about 45:1 to about 55:1 (w/w). In some aspects, the ratio of lipid to nucleic acid in the nanoparticle can be about 50:1 (w/w).

In some aspects, including in the LNPs put forth in Table A, the ratio of lipid to nucleic acid in the nanoparticle can be about 50:1 to about 70:1 (w/w), or about 55:1 to about 65:1 (w/w). In some aspects, the ratio of lipid to nucleic acid in the nanoparticle can be about 60:1 (w/w).

In some aspects, including in the LNPs put forth in Table A, the ratio of lipid to nucleic acid in the nanoparticle can be about 70:1 to about 90:1 (w/w), or about 75:1 to about 85:1 (w/w). In some aspects, the ratio of lipid to nucleic acid in the nanoparticle can be about 80:1 (w/w).

In some aspects, including in the LNPs put forth in Table A, the ratio of lipid to nucleic acid in the nanoparticle can be about 90:1 to about 110:1 (w/w), or about 95:1 to about 105:1 (w/w). In some aspects, the ratio of lipid to nucleic acid in the nanoparticle can be about 100:1 (w/w).

In some aspects, including in the LNPs put forth in Table A, the ratio of lipid to nucleic acid in the nanoparticle can be about 110:1 to about 130:1 (w/w), or about 115:1 to about 125:1 (w/w). In some aspects, the ratio of lipid to nucleic acid in the nanoparticle can be about 120:1 (w/w).

In some aspects wherein the LNPs of the present disclosure comprise both RNA (e.g. mRNA) and DNA, the ratio of RNA to DNA (RNA:DNA) in the LNPs can be about 1:1, or about 2:1, or about 3:1, or about 4:1, or about 5:1, or about 6:1, or about 7:1, or about 8:1, or about 9:1 or about 10:1.

In some aspects, wherein the LNPs of the present disclosure comprise mRNA, gRNA and DNA, the ratio of mRNA to gRNA to DNA (mRNA:gRNA:DNA) can be about 1:1:1, about 2:1:1, about 3:1:1, about 4:1:1 or about 5:1:1.

In some aspects, wherein the LNPs of the present disclosure comprise mRNA, gRNA and DNA, the ratio of gRNA to mRNA to DNA (gRNA:mRNA:DNA) can be about 1:1:1, about 2:1:1, about 3:1:1, about 4:1:1 or about 5:1:1.

In some aspects, wherein the LNPs of the present disclosure comprise mRNA, gRNA and DNA, the ratio of DNA to gRNA to mRNA (DNA:gRNA:mRNA) can be about 1:1:1, about 2:1:1, about 3:1:1, about 4:1:1 or about 5:1:1.

Further characteristics of the nucleic acid molecules of the present disclosure are provided herein.

### Exemplary LNPs of the Present Disclosure

The following are exemplary LNPs of the present disclosure.

In some aspects, a lipid nanoparticle is provided comprising about 40.75% of at least one compound of Formula (I) by moles, about 51.75% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 30.75% to about 50.75% of at least one compound of Formula (I) by moles, about 41.75% to about 61.75% of at least one structural lipid by moles, about 0.1% to about 15% of at least one phospholipid by moles, and about 0.1% to about 12.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the present disclosure provides a lipid nanoparticle comprising about 35.75% to about 45.75% of at least one compound of Formula (I) by moles, about 46.75% to about 56.75% of at least one structural lipid by moles, about 1% to about 10% of at least one phospholipid by moles, and about 1% to about 7.5% of at least one PEGylated lipid by moles, wherein the lipid nanoparticle comprises at least one nucleic acid, wherein the at least one nucleic acid comprises at least one RNA molecule (e.g. mRNA molecule). In some aspects, the mRNA molecule further comprises a 5′-CAP. In some aspects, the ratio of lipid to nucleic acid in the nanoparticle can be about 50:1 to about 70:1 (w/w), or about 55:1 to about 65:1 (w/w). In some aspects, the ratio of lipid to nucleic acid in the nanoparticle can be about 60:1 (w/w).

In some aspects, the nucleic acid molecule is a DNA molecule. Thus, the present disclosure provides a lipid nanoparticle is provided comprising about 40.75% of at least one compound of Formula (I) by moles, about 51.75% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 30.75% to about 50.75% of at least one compound of Formula (I) by moles, about 41.75% to about 61.75% of at least one structural lipid by moles, about 0.1% to about 15% of at least one phospholipid by moles, and about 0.1% to about 12.5% of at least one PEGylated lipid by moles, wherein the lipid nanoparticle comprises at least one nucleic acid, wherein the at least one nucleic acid comprises at least one DNA molecule. In some aspects, the present disclosure provides a lipid nanoparticle comprising about 35.75% to about 45.75% of at least one compound of Formula (I) by moles, about 46.75% to about 56.75% of at least one structural lipid by moles, about 1% to about 10% of at least one phospholipid by moles, and about 1% to about 7.5% of at least one PEGylated lipid by moles, wherein the lipid nanoparticle comprises at least one nucleic acid, wherein the at least one nucleic acid comprises at least one DNA molecule. In one aspect, the at least one DNA molecule is a Doggy Bone DNA molecule. In some aspects, the at least one DNA molecule is a DNA nanoplasmid. In some aspects, the at least one nucleic acid further comprises at least one RNA molecule. In some aspects, the mRNA molecule further comprises a 5′-CAP. In some aspects, the ratio of lipid to nucleic acid in the nanoparticle can be about 10:1 to about 30:1 (w/w), or about 15:1 to about 25:1 (w/w). In some aspects, the ratio of lipid to nucleic acid in the nanoparticle can be about 20:1 (w/w).

In some aspects, a lipid nanoparticle comprising at least one nucleic acid can comprise at least one nucleic acid molecule, wherein the at least one nucleic acid molecule is at least one RNA molecule and at least one DNA molecule.

In some aspects, a lipid nanoparticle is provided comprising about 40.75% of at least one compound of Formula (I) by moles, about 51.75% of at least one structural lipid by moles, about 5% of at least one phospholipid by moles, and about 2.5% of at least one PEGylated lipid by moles, wherein the lipid nanoparticle comprises at least one nucleic acid, wherein the at least one nucleic acid comprises at least one RNA molecule and at least one DNA molecule. In some aspects, the present disclosure provides a lipid nanoparticle comprising about 30.75% to about 50.75% of at least one compound of Formula (I) by moles, about 41.75% to about 61.75% of at least one structural lipid by moles, about 0.1% to about 15% of at least one phospholipid by moles, and about 0.1% to about 12.5% of at least one PEGylated lipid by moles, wherein the lipid nanoparticle comprises at least one nucleic acid, wherein the at least one nucleic acid comprises at least one RNA molecule and at least one DNA molecule. In some aspects, the present disclosure provides a lipid nanoparticle comprising about 35.75% to about 45.75% of at least one compound of Formula (I) by moles, about 46.75% to about 56.75% of at least one structural lipid by moles, about 1% to about 10% of at least one phospholipid by moles, and about 1% to about 7.5% of at least one PEGylated lipid by moles, wherein the lipid nanoparticle comprises at least one nucleic acid, wherein the at least one nucleic acid comprises at least one RNA molecule and at least one DNA molecule. In one aspect, the at least one DNA molecule is a Doggy Bone DNA molecule. In some aspects, the at least one DNA molecule is a DNA nanoplasmid. In some aspects, the mRNA molecule further comprises a 5′-CAP. In some aspects, the ratio of lipid to nucleic acid in the nanoparticle can be about 10:1 to about 30:1 (w/w), or about 15:1 to about 25:1 (w/w). In some aspects, the ratio of lipid to nucleic acid in the nanoparticle can be about 20:1 (w/w). In some aspects, the at least one nucleic acid comprises at least one RNA molecule and at least one DNA molecule in a 1:2 ratio.

### Pharmaceutical Compositions of the Present Disclosure

In some aspects, the present disclosure provides a pharmaceutical composition comprising at least one lipid nanoparticle of the present disclosure. In some aspects, the present disclosure provides a pharmaceutical composition comprising at least one first nanoparticle of the present disclosure and at least one second nanoparticle of the present disclosure, wherein the at least one first nanoparticle comprises at least one nucleic acid molecule encoding at least one transposase, wherein the at least one second nanoparticle comprises at least one nucleic acid molecule encoding at least one transposon. In some aspects, the at least one nucleic acid molecule encoding at least one transposase can be an RNA molecule (e.g. mRNA molecule) and the at least one nucleic acid molecule encoding at least one transposon can be a DNA molecule (e.g. a Doggy Bone DNA molecule or a DNA nanoplasmid).

In some aspects, the present disclosure provides a composition comprising at least one cell that has been contacted by at least one nanoparticle of the present disclosure. In some aspects, the present disclosure provides a composition comprising at least one cell that has been genetically modified using at least one nanoparticle of the present disclosure. In some aspects, the present disclosure provides a composition comprising at least one cell that has been genetically modified using any method of the present disclosure.

In some aspects, the present disclosure provides a pharmaceutical composition comprising at least one cell that has been contacted by at least one nanoparticle of the present disclosure. In some aspects, the present disclosure provides a pharmaceutical composition comprising at least one cell that has been genetically modified using at least one nanoparticle of the present disclosure. In some aspects, the present disclosure provides a pharmaceutical composition comprising at least one cell that has been genetically modified using any method of the present disclosure.

### Methods of the Present Disclosure

The present disclosure provides a method of delivering at least one nucleic acid to at least one cell comprising contacting the at least one cell with at least one composition of the present disclosure. The present disclosure provides a method of delivering at least one nucleic acid to at least one cell comprising contacting the at least one cell with at least one nanoparticle of the present disclosure.

In all methods, compositions and kits of the present disclosure, at least one cell can be a lung cell. A lung cell can include, but is not limited to, endothelial cells, epithelial cells, and leukocytes.

In some aspects of any methods of the present disclosure, a cell can be in vivo, ex vivo or in vitro. In some aspects, any of the methods of the present disclosure can be applied in vivo, ex vivo or in vitro.

The present disclosure provides a method of genetically modifying at least one cell comprising contacting the at least one cell with at least one composition of the present disclosure. The present disclosure provides a method of genetically modifying at least one cell comprising contacting the at least one cell with at least one nanoparticle of the present disclosure.

In some aspects, genetically modifying a cell can comprise delivering at least one exogenous nucleic acid to the cell such that the cell expresses at least one protein that the cell otherwise would not normally express, or such that the at least one cell expresses at least one protein at a level that is higher than the level that the cell would otherwise normally express the at least one protein, or such that the cell expresses at least one protein at a level that is lower than the level that the cell would otherwise normally express. In some aspects, genetically modifying a cell can comprise delivering at least one exogenous nucleic to the cell such that at least one exogenous nucleic acid is integrated into the genome of the at least one cell.

In some aspects, the methods of the present disclosure can yield a plurality of cells, wherein at least about 1%, or at least about 2%, or at least about 3%, or at least about 4%, or at least about 5%, or at least 10%, or at least 15%, or at least 20%, or at least 25%, or at least 30%, or at least 35%, or at least 40%, or at least 45%, or at least 50%, or at least 55%, or at least 60%, or at least about 65%, or at least about 70%, or at least about 75%, or at least about 80%, or at least about 85%, or at least about 90%, or at least about 95%, or at least about 99% of the cell in the plurality express at least one protein that was encoded in at least one nucleic acid that was delivered to the plurality of cells via a nanoparticle of the present disclosure.

The present disclosure provides a method of treating at least one disease in a subject, the method comprising administering to the subject at least one therapeutically effective amount of at least one composition of the present disclosure comprising at least one nucleic acid encoding a therapeutic protein.

The present disclosure provides a method of treating at least one disease in a subject, the method comprising administering a therapeutically effective amount of at least one nanoparticle of the present disclosure comprising at least one nucleic acid encoding a therapeutic protein.

The present disclosure provides a method of treating at least one disease in a subject, the method comprising administering a therapeutically effective amount of cells, wherein the cells have been contacted by at least one nanoparticle of the present disclosure comprising at least one nucleic acid encoding a therapeutic protein. The present disclosure provides a method of treating at least one disease in a subject, the method comprising administering a therapeutically effective amount of cells, wherein the cells have been genetically modified using the compositions and/or methods of the present disclosure.

In some aspects, the at least one disease can be a malignant disease, including, but not limited to, cancer. In some aspects, the at least one disease can be a lung disease or disorder. In some aspects, the at least one disease can be cystic fibrosis.

Accordingly, the present disclosure provides a method of treating a lung disease or disorder in a subject in need thereof comprising administering to the subject at least one composition comprising at least one lipid nanoparticle of the present disclosure.

Accordingly, the present disclosure provides a method of treating cystic fibrosis in a subject in need thereof comprising administering to the subject at least one composition comprising at least one lipid nanoparticle of the present disclosure.

The present disclosure provides a method of preferential delivery of at least one composition comprising at least one lipid nanoparticle of the present disclosure to a lung or a lung cell in a subject in need thereof, the method comprising administering to the subject the at least one composition, thereby providing a greater amount, expression or activity of the at least one composition in the lung or the lung cell of the subject as compared to that achieved in a non-lung organ or a non-lung cell in the subject.

### Nucleic Acid Molecules

In some aspects, a nucleic acid molecule can be an RNA molecule. Thus, in some aspects, a lipid nanoparticle can comprise at least one RNA molecule. The at least one RNA molecule can be encapsulated within the lipid nanoparticle. In some aspects, an RNA molecule can be an mRNA molecule. In some aspects, a lipid nanoparticle can comprise at least one mRNA molecule. The mRNA molecule can be encapsulated within the lipid nanoparticle.

In some aspects, a nucleic acid molecule can be a synthetic nucleic acid molecule. In some aspects, a nucleic acid molecule can be a non-naturally occurring nucleic acid molecule. In some aspects, a non-naturally occurring nucleic acid molecule can comprise at least one non-naturally occurring nucleotide. The at least one non-naturally occurring nucleotide can be any non-naturally occurring nucleotide known in the art. In some aspects, a nucleic acid molecule can be a modified nucleic acid molecule. In some aspects, a modified nucleic acid molecule can comprise at least one modified nucleotide. The at least one modified nucleotide can be any modified nucleic acid known in the art.

In some aspects, an mRNA molecule can be capped using any method and/or capping moiety known in the art. An mRNA molecule can be capped with m7G(5)ppp(5′)G moiety. A m7G(5′)ppp(5′)G moiety is also referred to herein as a “Cap0”. An mRNA molecule can be capped with a CleanCap® moiety. A CleanCap® moiety can comprise a m7G(5′)ppp(5′)(2′OMeA) (CleanCap® AG) moiety. A CleanCap® moiety can comprise a m7G(5′)ppp(5′)(2′OMeG) (CleanCap® GG) moiety. An mRNA molecule can be capped with an anti-reverse cap analog (ARCA®) moiety. An ARCA® moiety can comprise a m7(3′-O-methyl)G(5′)ppp(5′)G moiety. An mRNA molecule can be capped with a CleanCap® 3′OMe moiety (CleanCap®+ARCA®).

In some aspects, an mRNA molecule can comprise at least one modified nucleic acid.

The at least one modified nucleic acid can comprise 5-methoxyuridine (5moU). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70) %, or at least about 75%, or at least about 80%, or at least about 85%, at least about 90%, or at least about 95%, or at least about 99% of the uridine bases in an mRNA molecule are 5-methoxyuridine bases. In some aspects, all of the uridine bases in an mRNA molecule are 5-methoxyuridine bases. Without wishing to be bound by theory, 5-methoxyuridine can improve protein expression and reduce immunogenicity (see Li et al., Bioconjugate Chem. 2016, 27, 3, 849-853 and Vaidyanathan et al. Molecular Therapy—Nucleic Acids. 2018, 12, 530-542).

In some aspects, an mRNA molecule can comprise at least one modified nucleic acid.

The at least one modified nucleic acid can comprise N₁-methylpseudouridine (me¹Ψ). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80) %, or at least about 85%, at least about 90%, or at least about 95%, or at least about 99% of the uridine bases in an mRNA N₁-methylpseudouridine bases. In some aspects, all of the uridine bases in an mRNA molecule are N₁-methylpseudouridine bases. Without wishing to be bound by theory, N₁-methylpseudouridine can improve protein expression (see Li et al., Bioconjugate Chem. 2016, 27, 3, 849-853).

In some aspects, an mRNA molecule can comprise at least one modified nucleic acid.

The at least one modified nucleic acid can comprise pseudouridine (Ψ). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the uridine bases in an mRNA pseudouridine bases. In some aspects, all of the uridine bases in an mRNA molecule are pseudouridine bases. Without wishing to be bound by theory, pseudouridine can improve protein expression and reduce immunogenicity (see Li et al., Bioconjugate Chem. 2016, 27, 3, 849-853 and Vaidyanathan et al. Molecular Therapy—Nucleic Acids. 2018, 12, 530-542).

In some aspects, an mRNA molecule can comprise at least one modified nucleic acid.

The at least one modified nucleic acid can comprise 5-methylcytidine (5-MeC). In some aspects, at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, at least about 90%, or at least about 95%, or at least about 99% of the cytidine bases in an mRNA 5-MeC bases. In some aspects, all of the cytidine bases in an mRNA molecule are 5-MeC bases.

In some aspects, a nucleic acid molecule can comprise a DNA molecule. Thus, in some aspects, a lipid nanoparticle can comprise a DNA molecule. In some aspects, the DNA molecule can be a circular DNA molecule, such as, but not limited to, a DNA plasmid or DNA nanoplasmid. Thus, in some aspects, a lipid nanoparticle can comprise a circular DNA molecule. In some aspects, a lipid nanoparticle can comprise a Doggybone DNA molecule. In some aspects, a lipid nanoparticle can comprise a DNA plasmid. In some aspects, a lipid nanoparticle can comprise a DNA nanoplasmid. In some aspects, a DNA molecule can be a linearized DNA molecule, such as, but not limited to, a linearized DNA plasmid or a linearized DNA nanoplasmid.

A DNA plasmid or DNA nanoplasmid can comprise can be at least about 0.25 kb, or at least about 0.5 kb, or at least about 0.75 kb, or at least about 1.0 kb, or at least about 1.25 kb, or at least about 1.5 kb, or at least about 1.75 kb, or at least about 2.0 kb, or at least about 2.25 kb, or at least about 2.5 kb, or at least about 2.75 kb, or at least about 3.0 kb, or at least about 3.25 kb, or at least about 3.5 kb, or at least about 3.75 kb, or at least about 4.0 kb, or at least about 4.25 kb, or at least about 4.5 kb, or at least about 4.75 kb, or at least about 5.0 kb, or at least about 5.25 kb, or at least about 5.5 kb, or at least about 5.75 kb, or at least about 6.0 kb, or at least about 6.25 kb, or at least about 6.5 kb, or at least about 6.75 kb, or at least about 7.0 kb, or at least about 7.25 kb, or at least about 7.5 kb, or at least about 7.75 kb, or at least about 8.0 kb, or at least about 8.25 kb, or at least about 8.5 kb, or at least about 8.75 kb, or at least about 9.0 kb, or at least about 9.25 kb, or at least about 9.5 kb, or at least about 9.75 kb, or at least about 10.0 kb, or at least about 10.25 kb, or at least about 10.5 kb, or at least about 10.75 kb, or at least about 11.0 kb, or at least about 11.25 kb, or at least about 11.5 kb, or at least about 11.75 kb, or at least about 12 kb, or at least about 12.25 kb, or at least about 12.5 kb, or at least about 12.75 kb, or at least about 13.0 kb, or at least about 13.25 kb, or at least about 13.5 kb, or at least about 13.75 kb, or at least about 14.0 kb, or at least about 14.25 kb, or at least about 14.5 kb, or at least about 14.75 kb or at least about 15.0 kb in length.

In some aspects, a nucleic acid molecule formulated in a lipid nanoparticle of the present disclosure can comprise at least one transgene sequence. In some aspects, a transgene sequence can comprise a nucleotide sequence encoding at least one therapeutic protein. In some aspects, a transgene sequence can comprise a nucleotide sequence encoding at least one transposase. In some aspects, a transgene sequence can comprise a nucleotide sequence encoding at least one transposon. In some aspects, a transposon can comprise a nucleotide sequence encoding at least one therapeutic protein. In some aspects, a transposon can comprise a nucleotide sequence encoding at least one therapeutic protein and at least one protomer sequence, wherein the at least one therapeutic protein is operatively linked to the at least one promoter sequence.

In some aspects, the lipid nanoparticles of the present disclosure can be produced using a microfluidic-mixing platform. In some aspects, the microfluidic-mixing platform can be a non-turbulent microfluidic mixing platform.

In some aspects, a microfluidic-mixing platform can produce the lipid nanoparticles of the present invention by combining a miscible solvent phase comprising the lipid components of the nanoparticle and an aqueous phase comprising the lipid nanoparticle cargo (e.g. nucleic acid, DNA, mRNA, etc.) using a microfluidic device. In some aspects, the miscible solvent phase and the aqueous phase are mixed in the microfluidic device under laminar flow conditions that do not allow for immediate mixing of the two phases. As the two phases move under laminar flow in a microfluidic channel, microscopic features in the channel can allow for controlled, homogenous mixing to produce the lipid nanoparticles of the present disclosure.

In some aspects, the microfluidic-mixing platform can include, but are not limited to the NanoAssemblr® Spark (Precision NanoSystems), the NanoAssemblr® Ignite™ (Precision NanoSystems), the NanoAssemblr® Benchtop (Precision NanoSystems), the NanoAssemblr® Blaze (Precision NanoSystems) or the NanoAssemblr® GMP System (Precision NanoSystems).

In some aspects, the lipid nanoparticles of the present disclosure can be produced using a microfluidic-mixing platform, wherein the microfluidic mixing platform mixes at a rate of at least about 2.5 ml/min, or at least about 5 ml/min, or at least about 7.5 ml/min, or at least about 10 ml/min, or at least about 12.5 ml/min, or at least about 15 ml/min, or at least about 17.5 ml/min, or at least about 20 ml/min, or at least about 22.5 ml/min, or at least about 25 ml/min, or at least about 27.5 ml/min, or at least about 30 ml/min.

In some aspects, the lipid nanoparticles of the present disclosure can be produced using a microfluidic-mixing platform, wherein the microfluidic mixing platform mixes a miscible solvent phase and an aqueous phase at a ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10, solvent:aqueous, v:v.

piggyBac ITR Sequences

In some aspects, a nucleic acid can comprise a piggyBac ITR sequence. In some aspects, a nucleic acid can comprise a first piggyBac ITR sequence and a second piggyBac ITR sequence.

In some aspects, a piggyBac ITR sequence can comprise any piggyBac ITR sequence known in the art.

In some aspects of the methods of the present disclosure, a piggyBac ITR sequence, such as a first piggyBac ITR sequence and/or a second piggyBac ITR sequence in an AAV piggyBac transposon can comprise, consist essentially of, or consist of a Sleeping Beauty transposon ITR, a Helraiser transposon ITR, a Tol2 transposon ITR, a TcBuster transposon ITR or any combination thereof.

### Transposition Systems

In some aspects, a nucleic acid can comprise a transposon or a nanotransposon comprising: a first nucleic acid sequence comprising: (a) a first inverted terminal repeat (ITR) or a sequence encoding a first ITR. (b) a second ITR or a sequence encoding a second ITR, and (c) an intra-ITR sequence or a sequence encoding an intra-ITR, wherein the intra-ITR sequence comprises a transposon sequence or a sequence encoding a transposon.

In some aspects, a nucleic acid can comprise a transposon or a nanotransposon comprising: a first nucleic acid sequence comprising: (a) a first inverted terminal repeat (ITR) or a sequence encoding a first ITR, (b) a second ITR or a sequence encoding a second ITR, and (c) an intra-ITR sequence or a sequence encoding an intra-ITR, wherein the intra-ITR sequence comprises a transposon sequence or a sequence encoding a transposon, and a second nucleic acid sequence comprising an inter-ITR sequence or a sequence encoding an inter-ITR, wherein the length of the inter-ITR sequence is equal to or less than 700 nucleotides.

The transposon or nanotransposon of the present disclosure can be a piggyBac™ (PB) transposon. In some aspects when the transposon is a PB transposon, the transposase is a piggyBac™ (PB) transposase a piggyBac-like (PBL) transposase or a Super piggyBac™ (SPB) transposase. Preferably, the sequence encoding the SPB transposase is an mRNA sequence.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3, 5′-TTCT-3′ and 5′-TTTT-3′. The PB or PBL transposon system has no payload limit for the genes of interest that can be included between the ITRs.

Exemplary amino acid sequences for one or more PB, PBL and SPB transposases are disclosed in U.S. Pat. Nos. 6,218,185; 6,962,810 and 8,399,643. In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 1.

The PB or PBL transposase can comprise or consist of an amino acid sequence having an amino acid substitution at two or more, at three or more or at each of positions 30, 165, 282, and/or 538 of the sequence of SEQ ID NO: 1. The transposase can be a SPB transposase that comprises or consists of the amino acid sequence of the sequence of SEQ ID NO: 1 wherein the amino acid substitution at position 30 can be a substitution of a valine (V) for an isoleucine (I), the amino acid substitution at position 165 can be a substitution of a serine(S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 2.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 1 or SEQ ID NO: 2 are described in more detail in PCT Publication No. WO 2019/173636 and PCT/US2019/049816.

In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 3.

The PB or PBL transposase can comprise or consist of an amino acid sequence having an amino acid substitution at two or more, at three or more or at each of positions 29, 164, 281, and/or 537 of the sequence of SEQ ID NO: 3. The transposase can be a SPB transposase that comprises or consists of the amino acid sequence of the sequence of SEQ ID NO: 3 wherein the amino acid substitution at position 29 can be a substitution of a valine (V) for an isoleucine (I), the amino acid substitution at position 164 can be a substitution of a serine(S) for a glycine (G), the amino acid substitution at position 281 can be a substitution of a valine (V) for a methionine (M), and the amino acid substitution at position 537 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 4.

In certain aspects wherein the transposase comprises the above-described mutations at positions 29, 164, 281, and/or 537, the PB, PBL and SPB transposases can further comprise an amino acid substitution at one or more of positions 2, 45, 81, 102, 118, 124, 176, 179, 184, 186, 199, 206, 208, 225, 234, 239, 240, 242, 257, 295, 297, 310, 314, 318, 326, 327, 339, 420, 435, 455, 469, 485, 502, 551, 569 and 590 of the sequence of SEQ ID NO: 3 or SEQ ID NO: 4 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.A transposon or nanotransposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180) or a hyperactive Sleeping Beauty (SB100×) transposase.

A transposon or nanotransposon of the present disclosure can be a Helraiser transposon. An exemplary Helraiser transposon includes Helibat1. In some aspects, when the transposon is a Helraiser transposon, the transposase is a Helitron transposase (for example, as disclosed in WO 2019/173636).

A transposon or nanotransposon of the present disclosure can be a Tol2 transposon. In some aspects, when the transposon is a Tol2 transposon, the transposase is a Tol2 transposase (for example, as disclosed in WO 2019/173636).

A transposon or nanotransposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636). The TcBuster transposase can comprise or consist of a naturally occurring amino acid sequence or a non-naturally occurring amino acid sequence. The polynucleotide encoding a TcBuster transposase can comprise or consist of a naturally occurring nucleic acid sequence or a non-naturally occurring nucleic acid sequence.

In some aspects, a mutant TcBuster transposase comprises one or more sequence variations when compared to a wild type TcBuster transposase as described in more detail in PCT Publication No. WO 2019/173636 and PCT/US2019/049816.

The cell delivery compositions (e.g., transposons) disclosed herein can comprise a nucleic acid molecule encoding a therapeutic protein or therapeutic agent. Examples of therapeutic proteins include those disclosed in PCT Publication No. WO 2019/173636 and PCT/US2019/049816.

### Gene Editing Systems

The present disclosure provides a gene editing composition and/or a cell comprising the gene editing composition. The gene editing composition can comprise a nanoparticle comprising a nucleic acid, wherein the nucleic acid comprises a sequence encoding a DNA binding domain and a sequence encoding a nuclease protein or a nuclease domain thereof. The sequence encoding a nuclease protein or the sequence encoding a nuclease domain thereof can comprise a DNA sequence, an RNA sequence, or a combination thereof. The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. In some aspects, the fusion protein can further comprise at least one nuclear localization signal (NLS). In some aspects, the fusion protein can further comprise at least two NLSs. The gene editing composition can further comprise a guide sequence. The guide sequence can comprise an RNA sequence.

A transgene can comprise a nucleic sequence encoding a small, Cas9 (Cas9) operatively-linked to an effector. The disclosure provides a fusion protein comprising, consisting essentially of or consisting of a DNA localization component and an effector molecule, wherein the effector comprises a small, Cas9 (Cas9). A small Cas9 construct of the disclosure can comprise an effector comprising a type IIS endonuclease.

A transgene can comprise a nucleic sequence encoding an inactivated, small, Cas9 (dSaCas9) operatively-linked to an effector. A transgene can comprise a nucleic sequence encoding a fusion protein comprising, consisting essentially of or consisting of a DNA localization component and an effector molecule, wherein the effector comprises a small, inactivated Cas9 (dSaCas9). A small, inactivated Cas9 (dSaCas9) construct of the disclosure can comprise an effector comprising a type IIS endonuclease.

A transgene can comprise a nucleic sequence encoding an inactivated Cas9 (dCas9) operatively-linked to an effector. A transgene can comprise a nucleic sequence encoding a fusion protein comprising, consisting essentially of or consisting of a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptoccocus pyogenes. The dCas9 can comprise a dCas9 with substitutions at amino acid positions 10 and 840, which inactivate the catalytic site. In some aspects, these substitutions are D10A and H840A.

A cell comprising the gene editing composition can express the gene editing composition stably or transiently. Preferably, the gene editing composition is expressed transiently. The guide RNA can comprise a sequence complementary to a target sequence within a genomic DNA sequence. The target sequence within a genomic DNA sequence can be a target sequence within a safe harbor site of a genomic DNA sequence.

Gene editing compositions, including Cas-CLOVER, and methods of using these compositions for gene editing are described in detail in U.S. Patent Publication Nos. 2017/0107541, 2017/0114149, 2018/0187185 and U.S. Pat. No. 10,415,024. In some aspects, a Cas-CLOVER protein can comprise, consist essentially of, or consist of an amino acid sequence at least 65%, 70%, 75%, 80%, 85%, 90%, 95%, 96%, 97%, 98%, 99% or 100% (or any percentage in between) identical to SEQ ID NO: 5.

Accordingly, the present disclosure provides any of the lipid nanoparticle compositions described herein, wherein the lipid nanoparticle comprises at least one genomic editing composition, wherein the at least one genomic editing composition comprises: a) a nucleic acid molecule comprising a nucleic acid sequence encoding a fusion protein, wherein the fusion protein comprises (i) an inactivated Cas9 (dCas9) protein or an inactivated nuclease domain thereof, (ii) a Clo051 protein or a nuclease domain thereof; and b) at least one gRNA molecule. In some aspects, the fusion protein can further comprise at least one NLS. In some aspects, the at least one genomic editing composition can comprise at least two species of gRNA molecules.

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the composition as well known in the art or as described herein.

For example, the disclosed LNP compositions of the present invention can further comprise a diluent. In some compositions, the diluent can be phosphate buffered saline (“PBS”).

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides: derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

The compositions can also include a buffer or a pH-adjusting agent: typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid: Tris, tromethamine hydrochloride, or phosphate buffers. Preferred buffers are organic acid salts, such as citrate. In some aspects, the buffer can include sucrose.

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement:” Hsieh, D. S., Eds., pp. 59-90 (Marcel Dekker, Inc. New York 1994.), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670)). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized.

Suitable dosages are well known in the art. See, e.g., Wells et al., eds., Pharmacotherapy Handbook, 2nd Edition, Appleton and Lange, Stamford, Conn. (2000); PDR Pharmacopoeia. Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

In aspects where the compositions to be administered to a subject in need thereof are modified cells as disclosed herein, the cells can be administered between about 1×10³ and 1×10¹⁵ cells; 1×10³ and 1×10¹⁵ cells, about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In an aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/04981.

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In an aspect, the subject is a mammal. Preferably, the subject is human. The terms “subject” and “patient” are used interchangeably herein.

The disclosure provides a method for modulating or treating at least one malignant disease or disorder in a cell, tissue, organ, animal or subject. Non-limiting examples of a malignant disease or disorder include cancer, lung diseases or disorders, and cystic fibrosis.

Any method can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In an aspect, the administration is systemic. Systemic administration can be any means known in the art and described in detail herein. Preferably, systemic administration is by an intravenous injection or an intravenous infusion. In an aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises the step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

### Construction of Nucleic Acids

The isolated nucleic acids of the disclosure can be made using (a) recombinant methods, (b) synthetic techniques. (c) purification techniques, and/or (d) combinations thereof, as well-known in the art.

The nucleic acids can conveniently comprise sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also relates to vectors that include isolated nucleic acid molecules of the disclosure and host cells that are genetically engineered with the recombinant vectors, as is well known in the art. See, e.g., Sambrook, et al., supra: Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22. CD20, CD33, CD52, or any combination thereof. Cell surface markers further include the suicide gene marker RQR8 (Philip B et al. Blood. 2014 Aug. 21; 124 (8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

Those of ordinary skill in the art are knowledgeable in the numerous expression systems available for expression of a nucleic acid molecule encoding a protein of the disclosure.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

In the chemical formulas shown herein, the marking indicates the position where a functional group bonds to another portion of a molecule. Definitions of specific functional groups and chemical terms are described in more detail below.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group,” as used herein, it is meant that a particular functional moiety. e.g., O, S, or N, is temporarily blocked so that a reaction can be carried out selectively at another reactive site in a multifunctional compound. In certain embodiments, a protecting group reacts selectively in good yield to give a protected substrate that is stable to the projected reactions: the protecting group should be selectively removable in good yield by readily available, preferably non-toxic reagents that do not attack the other functional groups: the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art. “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein. “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-18 aliphatic carbon atoms. In certain embodiments, the alkyl, alkenyl, and alkynyl groups employed in the invention contain 1-15 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkyl” as used herein refers to saturated, straight- or branched-chain aliphatic groups having from 1 to 18 carbon atoms. As such. “alkyl” encompasses C₁, C₂, C₃, C₄, C₅, C₆, C₇, C₈, C₉, C₁₀, C₁₁ and C₁₂ groups. Examples of alkyl groups include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term “alkenyl” refers to an unsaturated straight or, when applicable, branched chain aliphatic group with one or more carbon-carbon double bonds, having from 2 to 18 carbon atoms. As such, “alkenyl” encompasses C₂, C₃, C₄, C₅, C₆, C₇, C₈, C₉, C₁₀, C₁₁ and C₁₂ groups. Alkenyl groups include, for example, ethenyl, propenyl, butenyl, 1-methyl-2-buten-1-yl, and the like.

The term “alkynyl” refers to an unsaturated straight or, when applicable, branched chain aliphatic group with one or more carbon-carbon triple bonds, having from 2 to 18 carbon atoms. As such, “alkynyl” encompasses C₂, C₃, C₄, C₅, C₆, C₇, C₈, C₉, C₁₀, C₁₁ and C₁₂ groups. Representative alkynyl groups include ethynyl, 2-propynyl (propargyl), 1-propynyl, and the like.

As used herein, the term “aryl” group is a C₆-C₁₄ aromatic moiety comprising one to three aromatic rings, which is optionally substituted. As such, “aryl” includes C₆, C₇, C₈, C₉, C₁₀, C₁₁, C₁₂, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl.

As used herein, the term “cycloalkyl” as employed herein includes saturated and partially unsaturated cyclic hydrocarbon groups having 3 to 12 carbons. As such, “cycloalkyl” includes C₃, C₄, C₅, C₆, C₇, C₈, C₉, C₁₀, C₁₁ and C₁₂ cyclic hydrocarbon groups. Representative cycloalkyl groups include, without limitation, cyclopropyl, cyclobutyl, cyclopentyl, cyclopentenyl, cyclohexyl, cyclohexenyl, cycloheptyl, and cyclooctyl.

As used herein, the term “halo” or “halogen” refers to fluoro, chloro, bromo and iodo.

It will be understood that the compounds of any one of the Formulae disclosed herein and any pharmaceutically acceptable salts thereof, comprise stereoisomers, mixtures of stereoisomers, polymorphs of all isomeric forms of said compounds.

The term “independently selected” is used herein to indicate that the R groups can be identical or different.

The term “substituted,” whether preceded by the term “optionally” or not, and “substituent,” as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50) nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to an aspect hereof, the term “antibody hereof” in its broadest sense also covers such analogs. Generally, in such analogs, one or more amino acid residues may have been replaced, deleted and/or added, compared to the antibodies hereof as defined herein.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter. SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence: (ii) the complement of a referenced nucleotide sequence or portion thereof: (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables 1, 2, or 3 below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 1.

TABLE 1
Conservative Substitutions I

| Side chain characteristics | | Amino Acid |
|---|---|---|
| Aliphatic | Non-polar | GAPILVF |
| | Polar-uncharged | CSTMNQ |
| | Polar-charged | DEKR |

| | |
|---|---|
| Aromatic | HFWY |
| Other | NQDE |

Alternately, conservative amino acids can be grouped as described in Lehninger. (Biochemistry. Second Edition: Worth Publishers. Inc. NY. N.Y. (1975). pp. 71-77) as set forth in Table 2.

TABLE 2
Conservative Substitutions II

| Side Chain Characteristic | | Amino Acid |
|---|---|---|
| Non-polar (hydrophobic) | Aliphatic: | ALIVP |
| | Aromatic: | FWY |
| | Sulfur-containing: | M |
| | Borderline: | GY |
| Uncharged-polar | Hydroxyl: | STY |
| | Amides: | NQ |
| | Sulfhydryl: | C |
| | Borderline: | GY |

| | |
|---|---|
| Positively Charged (Basic): | KRH |
| Negatively Charged (Acidic): | DE |

Alternately, exemplary conservative substitutions are set out in Table 3.

TABLE 3
onservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

For the following Examples, Compound Numbers are assigned to compounds of Formula (I) of the present disclosure according to the following:

| COMPOUND | |
|---|---|
| NO. | Structure |
| 1 | 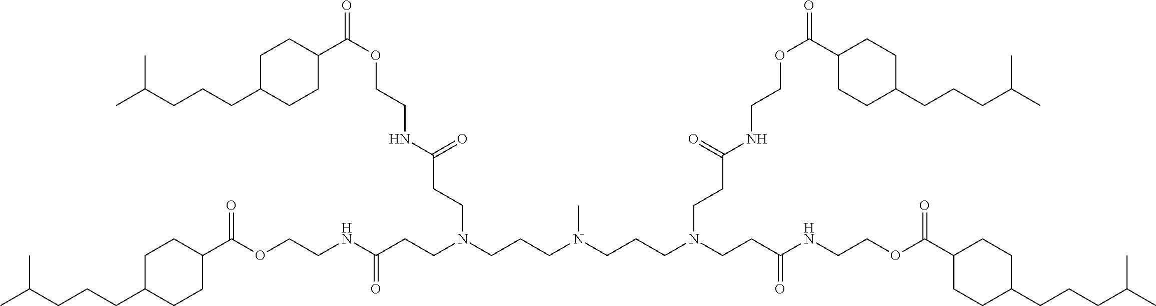 [SMILES: CC(C)CCCC1CCC(C(=O)OCCNC(=O)CCN(CCCN(C)CCCN(CCC(=O)NCCOC(=O)C2CCC(CCCC(C)C)CC2)CCC(=O)NCCOC(=O)C2CCC(CCCC(C)C)CC2)CCC(=O)NCCOC(=O)C2CCC(CCCC(C)C)CC2)CC1] |
| 2 | 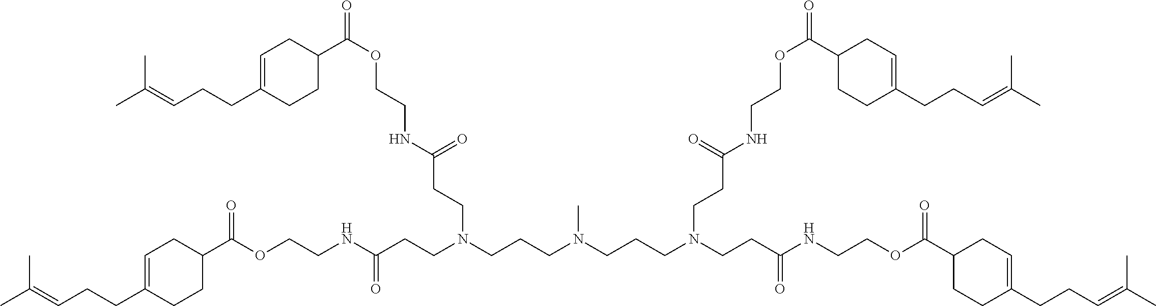 [SMILES: CC(C)=CCCC1=CCC(C(=O)OCCNC(=O)CCN(CCCN(C)CCCN(CCC(=O)NCCOC(=O)C2CC=C(CCC=C(C)C)CC2)CCC(=O)NCCOC(=O)C2CC=C(CCC=C(C)C)CC2)CCC(=O)NCCOC(=O)C2CC=C(CCC=C(C)C)CC2)CC1] |
| 3 | 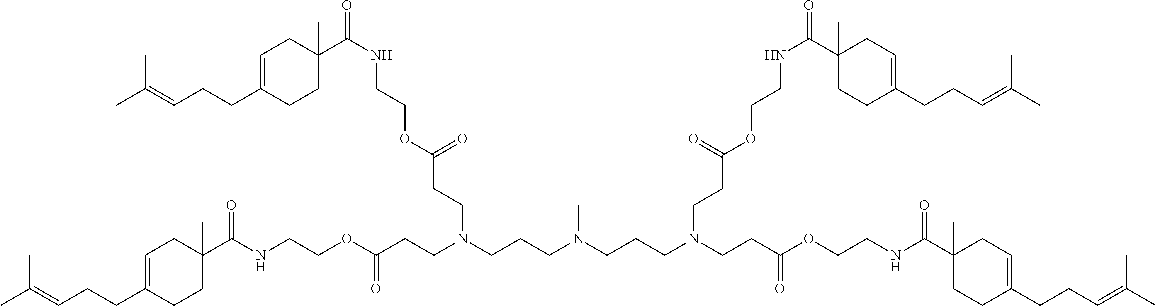 [SMILES: CC(C)=CCCC1=CCC(C)(C(=O)NCCOC(=O)CCN(CCCN(C)CCCN(CCC(=O)OCCNC(=O)C2(C)CC=C(CCC=C(C)C)CC2)CCC(=O)OCCNC(=O)C2(C)CC=C(CCC=C(C)C)CC2)CCC(=O)OCCNC(=O)C2(C)CC=C(CCC=C(C)C)CC2)CC1] |
| 4 | 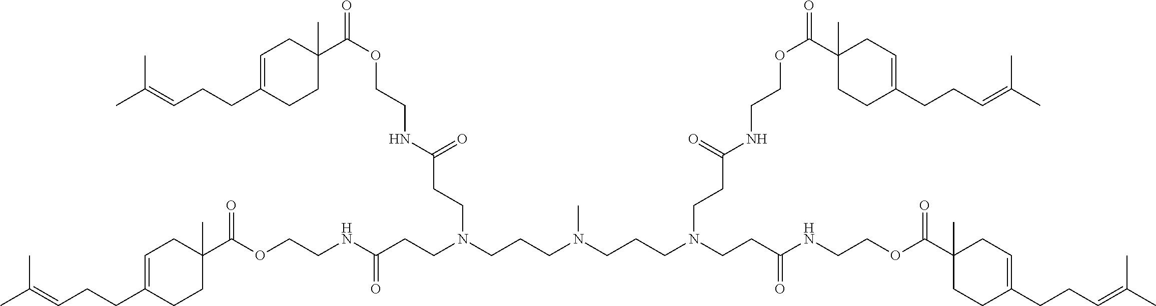 [SMILES: CC(C)=CCCC1=CCC(C)(C(=O)OCCNC(=O)CCN(CCCN(C)CCCN(CCC(=O)NCCOC(=O)C2(C)CC=C(CCC=C(C)C)CC2)CCC(=O)NCCOC(=O)C2(C)CC=C(CCC=C(C)C)CC2)CCC(=O)NCCOC(=O)C2(C)CC=C(CCC=C(C)C)CC2)CC1] |
| 5 | 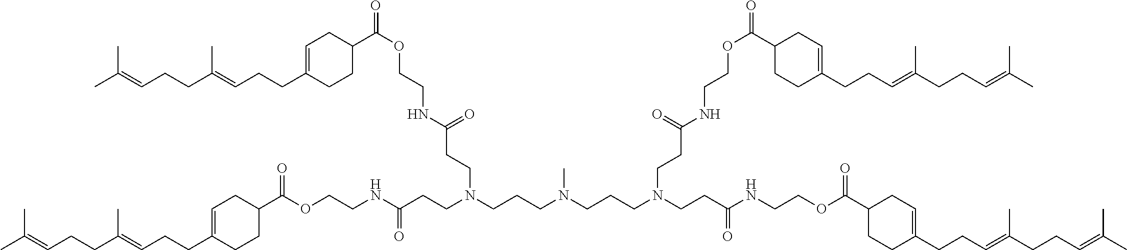 [SMILES: CC(C)=CCC/C(C)=C/CCC1=CCC(C(=O)OCCNC(=O)CCN(CCCN(C)CCCN(CCC(=O)NCCOC(=O)C2CC=C(CC/C=C(\C)CCC=C(C)C)CC2)CCC(=O)NCCOC(=O)C2CC=C(CC/C=C(\C)CCC=C(C)C)CC2)CCC(=O)NCCOC(=O)C2CC=C(CC/C=C(\C)CCC=C(C)C)CC2)CC1] |
| 6 | 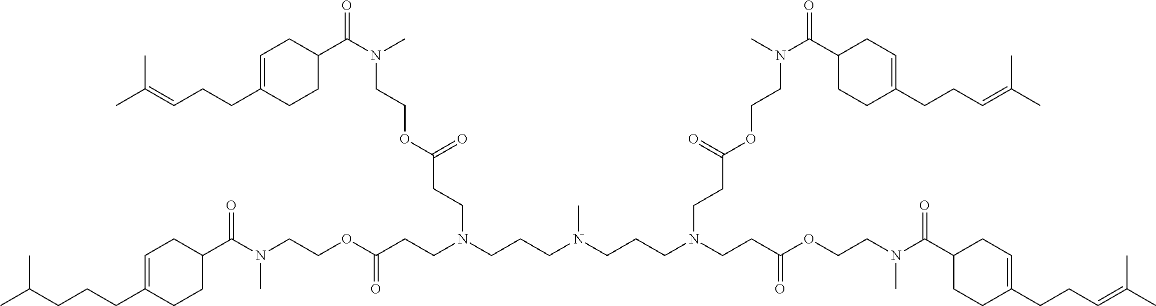 [SMILES: CC(C)=CCCC1=CCC(C(=O)N(C)CCOC(=O)CCN(CCCN(C)CCCN(CCC(=O)OCCN(C)C(=O)C2CC=C(CCC=C(C)C)CC2)CCC(=O)OCCN(C)C(=O)C2CC=C(CCCC(C)C)CC2)CCC(=O)OCCN(C)C(=O)C2CC=C(CCC=C(C)C)CC2)CC1] |
| 7 | 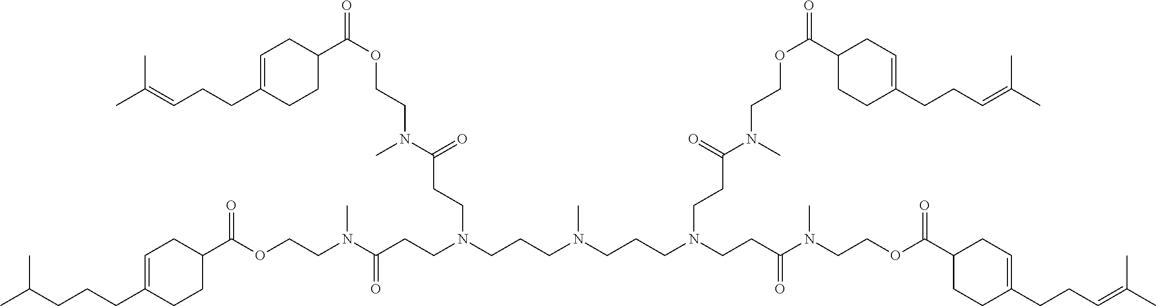 [SMILES: CC(C)=CCCC1=CCC(C(=O)OCCN(C)C(=O)CCN(CCCN(C)CCCN(CCC(=O)N(C)CCOC(=O)C2CC=C(CCC=C(C)C)CC2)CCC(=O)N(C)CCOC(=O)C2CC=C(CCCC(C)C)CC2)CCC(=O)N(C)CCOC(=O)C2CC=C(CCC=C(C)C)CC2)CC1] |

### Example 1—Preparation of COMPOUND NO. 1

COMPOUND NO. 1 was prepared in accordance with General Scheme A. The crude was purified by silica gel flash column chromatography with 5-8% MeOH/CH₂Cl₂. Pale brown oil, 32 mg, Yield: 31%; MS found 1383.1 [M+H]⁺, calcd for [C79H143N7O12=1382.08].

### Example 2—Preparation of COMPOUND NO. 2

COMPOUND NO. 2 was prepared in accordance with General Scheme A. The crude was purified by silica gel flash column chromatography with 8% MeOH/CH₂Cl₂. Pale brown oil, 0.76 g, Yield: 37%: ¹H NMR (499 MHz, CDCl₃) δ 5.43-5.34 (m, 4H), 5.13-5.03 (m, 4H), 4.20-4.08 (m, 8H), 3.58-3.42 (m, 8H), 3.16-3.00 (m, 4H), 2.74-1.79 (m, 71H), 1.69-1.56 (m, 28H).

### Example 3—Preparation of COMPOUND NO. 3

COMPOUND NO. 3 was prepared in accordance with General Scheme B2. The crude was purified by silica gel flash column chromatography with 8% MeOH/CH₂Cl₂. δ 6.23 (s, 4H), 5.43-5.35 (m, 4H), 5.07 (m, 4H), 4.15 (t, J=5.5 Hz, 8H), 3.53-3.44 (m, 8H), 2.75 (t, J=7.1 Hz, 8H), 2.44 (t, J=7.1 Hz, 12H), 2.39-2.25 (m, 4H), 2.17 (br, 3H), 2.10-2.03 (m, 10H), 2.00-1.93 (m, 16H), 1.91-1.84 (m, 6H), 1.68 (m, 12H), 1.62-1.48 (m, 24H), 1.17 (d, J=4.6 Hz, 12H). MS found 1424.3 [M+H]⁺, calcd for [C83H136N7O12=1423.0]

### Example 4—Preparation of COMPOUND NO. 4

COMPOUND NO. 4 was prepared in accordance with General Scheme B1. The crude was purified by silica gel flash column chromatography with 8% MeOH/CH₂Cl₂. ¹H NMR (499 MHz, CDCl₃) δ 5.33 (m, 4H), 5.06 (m, 4H), 4.14 (t, J=5.8 Hz, 8H), 3.48 (q, J=5.8 Hz, 8H), 2.71 (t, J=6.3 Hz, 8H), 2.52-2.43 (m, 8H), 2.34 (t, J=6.3 Hz, 8H), 2.25 (br, 3H), 2.07-2.01 (M, 8H), 2.00-1.79 (m, 24H), 1.67 (M, 16H), 1.60-1.57 (m, 20H), 1.18-1.17 (m, 12H). MS found 1423.4 [M+H]⁺, calcd for [C83H136N7O12=1423.0]

### Example 5—Preparation of COMPOUND NO. 5

COMPOUND NO. 5 was prepared in accordance with General Scheme A. The crude was purified by silica gel flash column chromatography with 30-50% MeOH/CH₂Cl₂ as eluants. Brown oil, 138 mg, Yield=61%: ¹H NMR (499 MHz, CDCl₃) δ 7.36-7.29 (m, 4H), 5.43-5.34 (m, 4H), 5.13-5.04 (m, 8H), 4.22-4.10 (m, 8H), 3.56-3.42 (m, 8H), 2.75-2.66 (m, 8H), 2.59-2.43 (m, 8H), 2.39-2.31 (m, 8H), 2.25-1.91 (m, 59H), 1.70-1.56 (m, 44H). Mass (ESI): C99H160N7O12 calculated 1639, Found 1640.

### Example 6—Preparation of COMPOUND NO. 6

COMPOUND NO. 6 was prepared in accordance with General Scheme C. ¹H NMR (500 MHz, CDCl₃): δ 5.42-5.43 (m, 8H), 5.08-5.11 (m, 8H), 4.23-4.17 (m, 8H), 3.71-3.58 (m, 8H), 3.13-0.91 (m, 12H), 2.71-2.65 (m, 12H), 2.45-2.38 (m, 12H), 2.35-2.25 (m, 8H), 2.27-2.15 (m, 3H), 2.14-2.01 (m, 16H), 2.02-193 (m, 6H), 1.8-1.67 (m, 12H), 1.68 (s, 12H), 1.60 (s, 12H). MS: m/z 1423 (M+1).

### Example 7—Preparation of COMPOUND NO. 7

COMPOUND NO. 7 was prepared in accordance with General Scheme C. ¹H NMR (500 MHZ, CDCl₃): δ 5.32-5.37 (m, 8H), 5.09-5.06 (m, 8H), 4.21-4.20 (m, 8H), 3.61-3.58 (m, 8H), 3.07-2.95 (m, 12H), 2.84-2.79 (m, 8H), 2.55-2.45 (m, 16H), 2.30-2.14 (m, 15H), 2.08-1.93 (m, 28H), 1.66 (s, 12H), 1.61 (s, 12H). MS: m/z 1423 (M+1).

### Example 8—LNPs of the Present Disclosure Deliver RNA with High Specificity to the Lungs In Vivo

The following is a nonlimiting example that provides exemplary methods for formulating a plurality of multi-component LNP compositions comprising exemplary compounds of Formula (I) and mRNA.

### A. Preparation

To formulate the LNPs, COMPOUND NO. 2, the phospholipid DOPC, the structural lipid cholesterol (Chol) and 1,2-dimyristoyl-sn-glycerol methoxypolyethylene glycol (DMG-PEG2000; Avanti Polar Lipids, Alabaster, Alabama, USA) were combined to prepare LNP compositions.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages. The LNP compositions are shown in Table 4.

TABLE 4

| COMPOUND | DOPC | Chol | PEG-DMG | Total | |
|---|---|---|---|---|---|
| NO._2 | (% | (% | (% | Lipid | Lipid:RNA |
| (% moles) | moles) | moles) | moles) | (mM) | (w/w) |
| 40.75 | 5 | 51.75 | 2.5 | 25 | 60 |
| 40.75 | 5 | 51.25 | 3 | 25 | 60 |

A 1 mg/ml solution of the 5′-CleanCap--fLuciferase mRNA (TriLink Biotech) to be incorporated into the LNPs was added to 150 mM sodium acetate buffer (pH 5.2) to form a stock solution and kept on ice. The lipid phase was mixed with the aqueous mRNA phase inside a microfluidic chip using a NanoAssemblr® instrument (Precision Nanosystems, Vancouver, BC, Canada) according to the manufacturer's instructions to form LNP compositions comprising encapsulated mRNAs. Nanoassemblr process parameters for mRNA encapsulation are shown in Table 5.

TABLE 5

| Total flow rate | Lipid phase:aqueous |
|---|---|
| (ml/min) | (RNA) phase (v/v) |
| 20 | 1:3 |

The resultant mRNA LNP compositions were then transferred to a Repligen Float-A-Lyzer dialysis device-having a molecular weight cut off (MWCO) of 8-10 kDa (Spectrum Chemical Mfg. Corp, CA, USA) and processed by dialysis against phosphate buffered saline (PBS) (dialysate:dialysis buffer volume at least 1:200 v/v), pH 7.4 overnight at 4° C. (or alternatively room temperature for at least 4 hours), to remove the 25% ethanol and achieve a complete buffer exchange. In some experiments the LNPs were further concentrated in an Amicon® Ultra-4 centrifugal filter unit, MWCO-30 kDa (Millipore Sigma, USA) spun at ˜4100×g in an ultracentrifuge. The mRNA LNPs were then stored at 4° C. until further use.

### B. In Vivo Screening

Adult BALB/C mice (n=3) were administered 0.5 mg/kg of total RNA formulated in an LNP composition listed in Table 4. One group of mice was treated with vehicle (PBS, Thermo Fisher Scientific, USA) as a negative control.

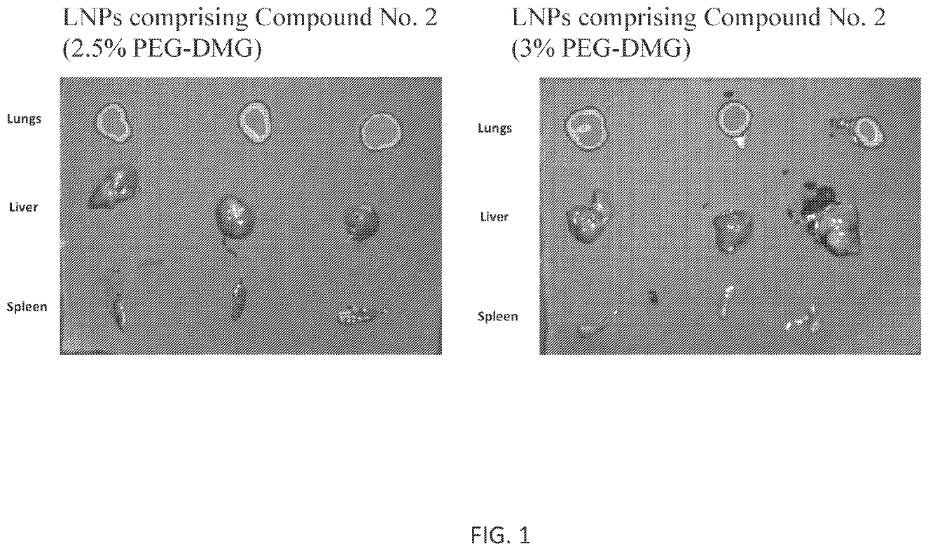

The location and extent of luciferase expression in treated and control mice were determined at 4 hr for RNA delivery by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed on the lungs, liver and spleen. BLI images are shown in FIG. 1 and quantified results are shown in Table 6.

TABLE 6

| | |
|---|---|
| LNPs comprising | Mean Luciferase Flux (p/s) |

| COMPOUND NO. 2 | Lungs | Liver | Spleen |
|---|---|---|---|
| 2.5% PEG-DMG | 1.28e9 | 3.70e6 | 8.75e6 |
| 3% PEG-DMG | 8.46e8 | 4.21e6 | 1.39e7 |

As shown in Table 6 and FIG. 1, LNP compositions of the present disclosure successfully delivered RNA to lung cells and the encoded transgene was expressed in the lung cells.

### Example 9—LNPs of the Present Disclosure Deliver DNA with High Specificity to the Lungs In Vivo

The following is a nonlimiting example that provides exemplary methods for formulating a plurality of multi-component LNP compositions comprising exemplary compounds of Formula (I) and DNA.

Adult BALB/C mice (n=3) were administered either (1) an LNP encapsulating firefly luciferase transposon (Nature Technology Corporation) or (2) a single co-encapsulated LNP encapsulating both firefly luciferase transposon and SPB. Mice receiving treatment with an LNP encapsulating luciferase transposon alone received 0.33 mg/kg or 0.5 mg/kg of LNP. Mice receiving treatment with a co-encapsulated LNP received 0.5 mg/kg or 0.75 mg/kg of co-encapsulated LNP encapsulating mRNA and DNA at 1:2 mRNA:DNA ratio. One group of mice was treated with vehicle (PBS, Thermo Fisher Scientific, USA) as a negative control.

The LNPS encapsulating firefly luciferase transposon were LNPs of the present disclosure comprising nanoplasmid DNA (SEQ ID NO: 6 or SEQ ID NO: 7) comprising a transposon, wherein the transposon comprised an expression cassette comprising, a first piggyBac inverted terminal repeat (right ITR), a nucleic acid sequence encoding for firefly luciferase, and a second piggyBac inverted terminal repeat (left ITR). The LNPs encapsulating firefly luciferase transposon comprised COMPOUND NO. 2, DOPC, Cholesterol and DMG-PEG2000 at the molar ratios shown in Table 7.

The single co-encapsulated LNP encapsulating both firefly luciferase transposon and SPB was an LNP of the present disclosure comprising the nanoplasmid DNA of SEQ ID NO: 6 or SEQ ID NO: 7, mRNA encoding active SPB and COMPOUND NO. 2, DOPC, Cholesterol and DMG-PEG2000 at the molar ratios shown in Table 7. All cytidine residues in the mRNA were 5-methylcytidine (5-MeC).

TABLE 7

| | | | | | Lipid:Nucleic |
|---|---|---|---|---|---|
| | | | | | Acid |
| COMPOUND | | | | Total | (Lipid:DNA or |
| NO. 2 | DOPC | Chol | PEG-DMG | Lipid | Lipid:RNA + DNA) |
| (% moles) | (% moles) | (% moles) | (% moles) | (mM) | (w/w) |
| 40.75 | 5.0 | 51.75 | 2.5 | 25 | 20 |

The location and extent of luciferase expression in treated and control mice were determined at 7 days post-injection for DNA delivery by bioluminescent imaging (BLI) of anesthetized mice using an IVIS Lumina in vivo imaging system (Perkin Elmer) according to the manufacturer's instructions. Briefly, mice were anesthetized using isoflurane in oxygen, and placed supine on a heated stage. Mice were then administered D-luciferin (Perkin-Elmer #122799) IP, and BLI was performed on the lungs, liver and spleen. The results of the BLI on the lungs are shown in Table 8 for the low dose treatment (0.33 mg/kg for luciferase transposon alone and 0.5 mg/kg for co-encapsulation treatment) and the high dose treatment (0.5 mg/kg for luciferase transposon alone and 0.75 mg/kg for co-encapsulation treatment).

TABLE 8

| | Treatment | | | |
|---|---|---|---|---|
| | Mean Luciferase Flux (p/s) | | | |
| | | Co- | | Co- |
| | Luciferase | encapsulated | Luciferase | encapsulated |
| | DNA (SEQ ID | LNP (SEQ ID | DNA (SEQ | LNP (SEQ ID |
| | NO: 6) | NO: 6 + SPB) | ID NO: 7) | NO: 7 + SPB) |
| Low dose | 9.49e4 | 1.86e5 | 1.18e5 | 3.43e5 |
| High dose | 1.01e5 | 3.92e6 | 1.62e5 | 4.90e6 |

As shown in Table 8, co-encapsulated LNPs (SPB mRNA+FLuc DNA at a 1:2 ratio) had significantly higher potency than the LNPs encapsulating FLuc DNA alone at the equivalent DNA dose, indicating that transposition facilitated by SPB was likely occurring. This example shows that LNP compositions of the present disclosure successfully delivered a two-component, DNA/RNA system to lung cells and the desired transgene was stably integrated into the genome and not episomal.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of Formula (I): 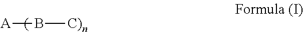
or a salt thereof,
wherein:
A is: 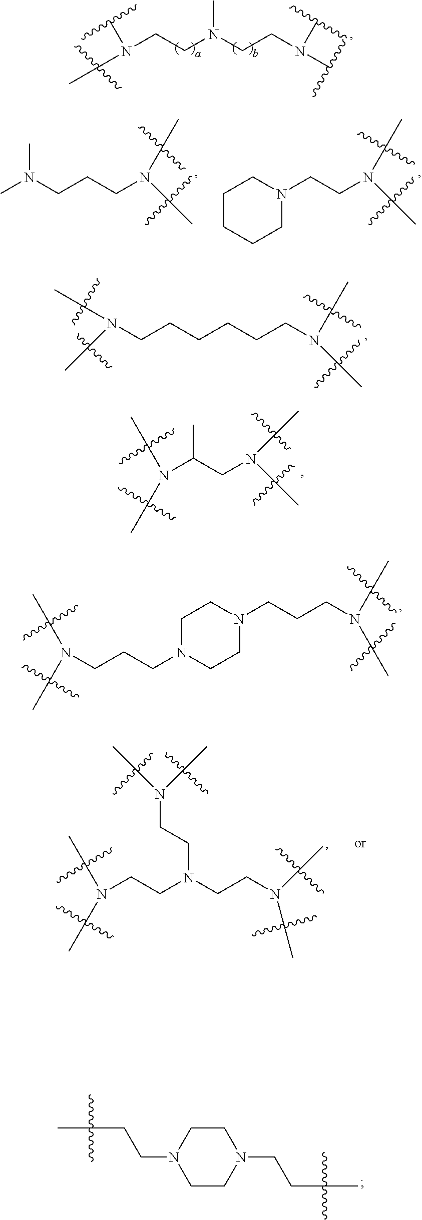
each B is independently: 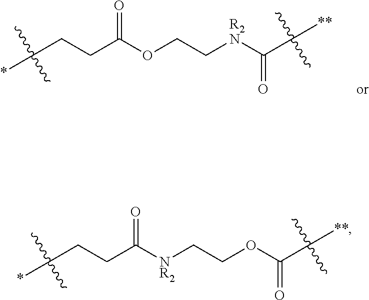
in which * indicates attachment to A and ** indicates attachment to C;
each C is independently: 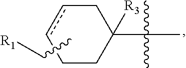
in which [symbol] denotes a single or double bond;
n is an integer between 2 to 6;
a is an integer between 1 to 5;
b is an integer between 1 to 5;
each R₁ is independently C₁-C₁₈ alkyl or C₂-C₁₈ alkenyl;
each R₂ is independently H or methyl; and
each R₃ is independently H or methyl.

2. The compound of claim 1, wherein A is:

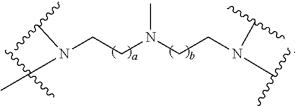

3. The compound of claim 1, wherein each B is: 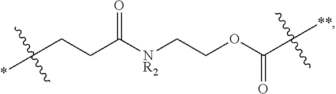
in which * indicates attachment to A and ** indicates attachment to C.

4. The compound of claim 1, wherein each B is: 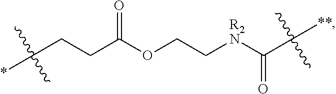
in which * indicates attachment to A and ** indicates attachment to C.

5. The compound of claim 1, wherein A is: 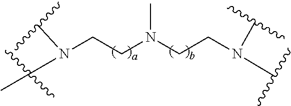
and each B is: 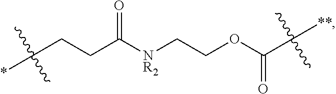
in which * indicates attachment to A and ** indicates attachment to C.

6. The compound of claim 1, wherein A is: 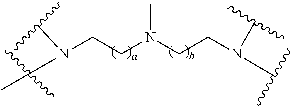
and each B is: 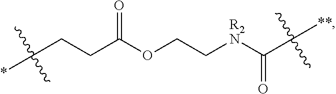
in which * indicates attachment to A and ** indicates attachment to C.

7.-10. (canceled)

11. The compound of claim 1, wherein each R₁ is C₂-C₁₈ alkenyl.

12.-13. (canceled)

14. The compound of claim 1, wherein each R₁ is C₁-C₁₈ alkyl.

15. (canceled)

16. The compound of claim 1, wherein (i) each R₃ is H and each R₁ is C₂-C₁₈ alkenyl; (ii) each R₃ is H and each R₁ is C₁-C₁₈ alkyl; (iii) each R₂ is H, each R₃ is H and each R₁ is C₂-C₁₈ alkenyl; or (iv) each R₂ is H, each R₃ is H and each R₁ is C₁-C₁₈ alkyl.

17.-24. (canceled)

25. The compound of claim 1, wherein a is 2; b is 2; or a is 2 and b is 2.

26. The compound of claim 1, wherein n is 4.

27. The compound of claim 1, wherein the compound is selected from a group consisting of:

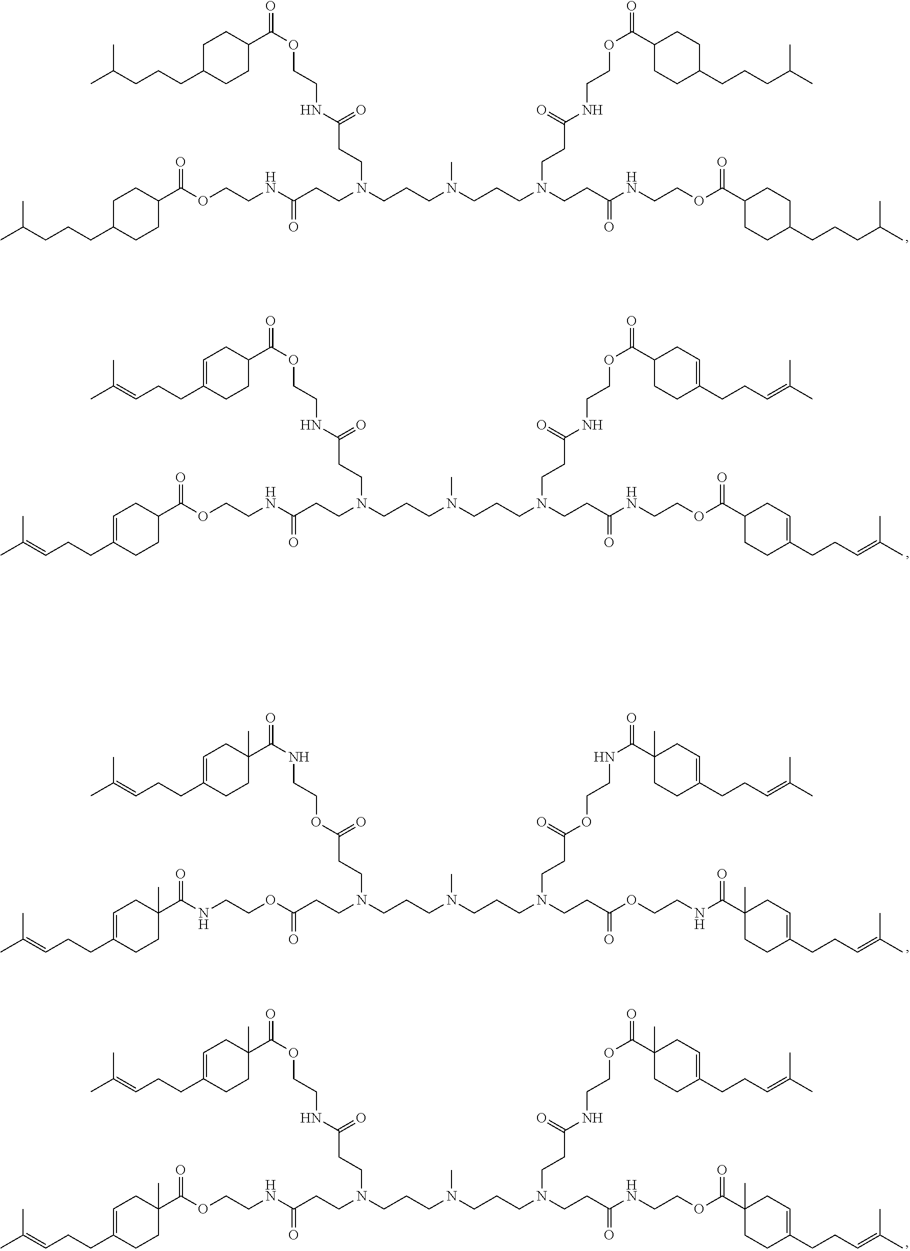

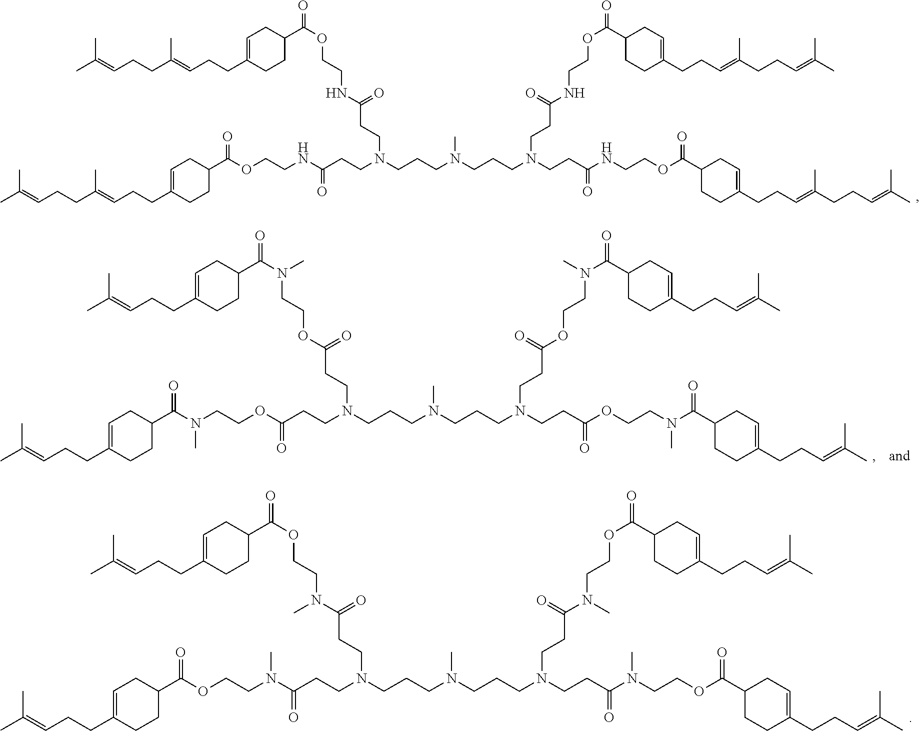

28. A composition comprising at least one lipid nanoparticle comprising at least one compound of claim 1.

29. The composition of claim 28, wherein the at least one lipid nanoparticle comprises about 40.75% of the at least one compound of Formula (I) by moles,
wherein the at least one nucleic acid molecule comprises: a) at least one RNA molecule, b) at least one DNA molecule, or c) a combination of at least one RNA and at least one DNA molecules,
wherein the at least one lipid nanoparticle further comprises:
about 51.75% of cholesterol by moles,
about 5% of DOPC by moles, and
about 2.5-3% of DMG-PEG2000 by moles; and
wherein the ratio of lipid to nucleic acid in the at least one nanoparticle is about 120:1, about 60:1 or about 20:1 (w/w).

30.-38. (canceled)

39. A pharmaceutical composition, comprising a composition of claim 28 and at least one pharmaceutically-acceptable excipient or diluent.

40. A method of delivering at least one nucleic acid to at least one cell comprising contacting the at least one cell with a composition of claim 28.

41. A method of genetically modifying at least one cell comprising contacting the at least one cell with a composition of claim 28.

42.-43. (canceled)

44. A method of treating at least one disease or disorder in a subject in need thereof comprising administering to the subject at least one therapeutically effective amount of the composition of claim 28.

45. The method of claim 44, wherein the at least one disease or disorder is a lung disease or disorder.

46. (canceled)

47. A method of preferential delivery of a composition to a lung or a lung cell in a subject in need thereof, the method comprising administering the composition of claim 28, thereby providing a greater amount, expression or activity of the composition in the lung or the lung cell of the subject as compared to that achieved in a non-lung organ or a non-lung cell in the subject.


SEQUENCE LISTING (7 sequences)

SEQ ID NO: 1 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 2 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 3 - protein, 593 aa, synthetic construct
GSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 4 - protein, 593 aa, synthetic construct
GSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 5 - protein, 1591 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 6 - DNA, 4521 bp, synthetic construct
  source 1..4521: mol_type = other DNA
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattcttattccctttgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgaagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 7 - DNA, 5313 bp, synthetic construct
  source 1..5313: mol_type = other DNA
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgtcgacgctagcatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgaagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta